(19) United States
(12) Patent Application Publication
(10) Pub. No.: US 2025/0032582 A1
(43) Pub. Date: Jan. 30, 2025

(54) COMPOSITIONS AND METHODS FOR CANCER IMMUNOTHERAPY

(71) Applicant: Mural Oncology, Inc., Waltham, MA (US)

(72) Inventors: Rita Dalal, Basking Ridge, NJ (US); Lei Sun, Waltham, MA (US); Bhaskar Rege, Sudbury, MA (US); Jessicca Rege, Sudbury, MA (US)

(73) Assignee: Mural Oncology, Inc., Waltham, MA (US)

(21) Appl. No.: 18/835,210

(22) PCT Filed: Feb. 10, 2023
(86) PCT No.: PCT/US23/62433

§ 371 Date: Aug. 1, 2024

Related U.S. Application Data
(60) Provisional application No. 63/309,199, filed on Feb. 11, 2022.

(51) Int. Cl. A61K 38/20; C07K 16/28

(52) U.S. Cl. CPC A61K 38/2013; C07K 16/2818; C07K 2317/24; C07K 2317/76

(57) ABSTRACT

The disclosure provides compositions and improved methods for the treatment of cancer comprising administering to a patient the fusion protein of SEQ ID NO: 1 at a less frequent dosing schedule (i.e., a single administration every three weeks or multiple administrations on non-consecutive days).

### RELATED APPLICATION

This application claims the benefit of U.S. Provisional Patent Application Ser. No. 63/309,199 filed Feb. 11, 2022, the entire disclosure of which is hereby incorporated herein by reference.

### BACKGROUND OF THE INVENTION

Interleukin-2 (IL-2) is a cytokine that induces proliferation of antigen-activated T cells and stimulates natural killer (NK) cells. The biological activity of IL-2 is mediated through a multi-subunit IL-2 receptor complex (IL-2R) of three polypeptide subunits that span the cell membrane: p55 (IL-2Rα, the alpha subunit, also known as CD25 in humans), p75 (IL-2R13, the beta subunit, also known as CD122 in humans) and p64 (IL-2Rγ, the gamma subunit, also known as CD132 in humans). T cell response to IL-2 depends on a variety of factors, including: (1) the concentration of IL-2; (2) the number of IL-2R molecules on the cell surface; and (3) the number of IL-2R occupied by IL-2 (i.e., the affinity of the binding interaction between IL-2 and IL-2R). The IL-2:IL-2R complex is internalized upon ligand binding and the different components undergo differential sorting. IL-2Rα is recycled to the cell surface, while IL-2 associated with the IL-2:IL-2Rβγ complex is routed to the lysosome and degraded.

Outcomes of systemic IL-2 administration in cancer patients are poor. While 15 to 20 percent of patients respond objectively to high-dose IL-2, the great majority do not, and many suffer severe, life-threatening side effects. Aldesleukin (recombinant human IL-2 (rhIL-2) also as known as Proleukin), is approved and used for the treatment of metastatic melanoma and RCC.

Among currently used therapies, rhIL-2 is one of the few treatment regimens that elicit a complete and durable response in a subset of patients, up to 12% in melanoma and 7% in RCC. High doses of rhIL-2 are required to stimulate cells that express the intermediate-affinity IL-2 receptor, including memory CD8⁺ T cells and natural killer (NK) cells, which are the primary cell types mediating anticancer immune responses.

It has been hypothesized that a contributing factor limiting the therapeutic efficacy of rhIL-2 is that it preferentially activates and induces the expansion of immunosuppressive CD4⁺ T_{regs}, which can counteract anticancer immune responses. This preferential activation is through binding of IL-2 to the high-affinity IL-2 receptor expressed on T_{regs}. Furthermore, it is hypothesized that direct interaction between rhIL-2 with high-affinity IL-2R expressed on vascular and pulmonary endothelial cells contributes to rhIL-2-mediated toxicity via capillary leak syndrome. Despite the poor tolerability of immunotherapy with rhIL-2, it remains one of the few treatment regimens for metastatic melanoma and RCC that elicits a complete and durable response in a subset of patients. Accordingly, new IL-2 therapies are needed to more effectively combat various cancers.

### SUMMARY OF THE INVENTION

The compositions, methods and treatment regimens in accordance with this disclosure provide numerous advantages for the treatment of cancer using IL-2 immunotherapy as compared to, for example, high dose rhIL-2 therapy (e.g., Aldesleukin).

In one aspect, the disclosure provides a method of treating cancer in a patient comprising administering to the patient a fusion protein comprising an amino acid sequence set forth in SEQ ID NO: 1, or a fusion protein comprising at least 80% identity to the amino acid sequence set forth in SEQ ID NO: 1, wherein the patient is administered at least a first dose of the fusion protein and a second dose of the fusion protein on non-consecutive days.

In certain embodiments, the fusion protein is administered at a dose of about 1 μg/kg to about 60 μg/kg.

In certain embodiments, the fusion protein is administered at a dose of about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 21 μg/kg, about 22 μg/kg, about 23 μg/kg, about 24 μg/kg, about 25 μg/kg, about 26 μg/kg, about 27 μg/kg, about 28 μg/kg, about 29 μg/kg, about 30 μg/kg, about 31 μg/kg, about 32 μg/kg, about 33 μg/kg, about 34 μg/kg, about 35 μg/kg, about 36 μg/kg, about 37 μg/kg, about 38 μg/kg, about 39 μg/kg, about 40 μg/kg, about 41 μg/kg, about 42 μg/kg, about 43 μg/kg, about 44 μg/kg, about 45 μg/kg, about 46 μg/kg, about 47 μg/kg, about 48 μg/kg, about 49 μg/kg, about 50 μg/kg, about 51 μg/kg, about 52 μg/kg, about 53 μg/kg, about 54 μg/kg, about 55 μg/kg, about 56 μg/kg, about 57 μg/kg, about 58 μg/kg, about 59 μg/kg, or about 60 μg/kg.

In certain embodiments, the fusion protein is administered at a dose of about 3 μg/kg, about 6 μg/kg, about 10 μg/kg, about 15 μg/kg, about 20 μg/kg, about 25 μg/kg, about 30 μg/kg, about 35 μg/kg, or about 40 μg/kg.

In certain embodiments, the first dose and the second dose are the same. In certain embodiments, the first dose and the second dose are different.

In certain embodiments, the patient is administered a third dose of the fusion protein.

In certain embodiments, the patient is administered the first dose of the fusion protein on day 1 and the second dose of the fusion protein on any one of day 3, day 4, day 5, day 6, day 7, day 8, day 9, day 10, day 11, day 12, day 13, day 14, day 15, day 16, day 17, day 18, day 19, day 20, or day 21.

In certain embodiments, the patient is administered the first dose of the fusion protein on day 1 and the second dose of the fusion protein on day 4.

In certain embodiments, the patient is administered the first dose of the fusion protein on day 1 and the second dose of the fusion protein on day 8.

In certain embodiments, the patient is administered the first dose of the fusion protein on day 1 and the second dose of the fusion protein on day 15.

In certain embodiments, the patient is administered the second dose of the fusion protein and the third dose of the fusion protein on consecutive days. In certain embodiments, the patient is administered the second dose of the fusion protein and the third dose of the fusion protein on non-consecutive days.

In certain embodiments, the patient is administered the third dose of the fusion protein 1 day, 2 days, 3 days, 4 days, 5 days, 6 days, 7 days, 8 days, 9 days, or 10 days after the second dose of the fusion protein.

In certain embodiments, the patient is administered the first dose of the fusion protein on day 1, the second dose of the fusion protein on any one of day 3, day 4, day 5, day 6, day 7, day 8, day 9, day 10, day 11, or day 12, and the third dose of the fusion protein on any one of day 13, day 14, day 15, day 16, day 17, day 18, day 19, day 20, or day 21.

In certain embodiments, the patient is administered the first dose of the fusion protein on day 1, the second dose of the fusion protein on any one of day 3, day 4, or day 5, and the third dose of the fusion protein on any one of day 6, day 7, day 8, day 9, or day 10.

In certain embodiments, the patient is administered the first dose of the fusion protein on day 1, the second dose of the fusion protein on any one of day 6, day 7, day 8, day 9, or day 10, and the third dose of the fusion protein on any one of day 13, day 14, day 15, day 16, or day 17.

In certain embodiments, the patient is administered the first dose of the fusion protein on day 1, the second dose of the fusion protein on day 4, and the third dose of the fusion protein on day 8.

In certain embodiments, the patient is administered the first dose of the fusion protein on day 1, the second dose of the fusion protein on day 8, and the third dose of the fusion protein on day 15.

In certain embodiments, the patient is administered a first dose of the fusion protein of about 10 μg/kg to about 30 μg/kg on day 1, and a second dose of the fusion protein of about 10 μg/kg to about 30 μg/kg on any one of day 3, day 4, day 5, day 6, day 7, day 8, day 9, day 10, day 11, day 12, day 13, day 14, day 15, day 16, day 17, day 18, day 19, day 20, or day 21.

In certain embodiments, the patient is administered a first dose of the fusion protein of about 10 μg/kg to about 15 μg/kg on day 1, and a second dose of the fusion protein of about 10 μg/kg to about 15 μg/kg on any one of day 3, day 4, day 5, or day 6.

In certain embodiments, the patient is administered a first dose of the fusion protein of about 10 μg/kg to about 25 μg/kg on day 1, and a second dose of the fusion protein of about 20 μg/kg to about 25 μg/kg on any one of day 6, day 7, day 8, day 9, or day 10.

In certain embodiments, the patient is administered a first dose of the fusion protein of about 15 μg/kg on day 1, and a second dose of the fusion protein of about 15 μg/kg on day 4.

In certain embodiments, the patient is administered a first dose of the fusion protein of about 20 μg/kg to about 25 μg/kg on day 1, and a second dose of the fusion protein of about 20 μg/kg to about 25 μg/kg on day 8.

In certain embodiments, the patient is administered a first dose of the fusion protein of about 6 μg/kg to about 25 μg/kg on day 1, a second dose of the fusion protein of about 6 μg/kg to about 25 μg/kg on any one of day 3, day 4, day 5, day 6, day 7, day 8, day 9, day 10, day 11, or day 12, and a third dose of the fusion protein of about 6 μg/kg to about 25 μg/kg on any one of day 13, day 14, day 15, day 16, day 17, day 18, day 19, day 20, or day 21.

In certain embodiments, the patient is administered a first dose of the fusion protein of about 6 μg/kg to about 15 μg/kg on day 1, a second dose of the fusion protein of about 6 μg/kg to about 15 μg/kg on any one of day 3, day 4, or day 5, and a third dose of the fusion protein of about 6 μg/kg to about 15 μg/kg on any one of day 6, day 7, day 8, day 9, or day 10.

In certain embodiments, the patient is administered a first dose of the fusion protein of about 10 μg/kg to about 20 μg/kg on day 1, a second dose of the fusion protein of about 10 μg/kg to about 20 μg/kg on any one of day 6, day 7, day 8, day 9, or day 10, and a third dose of the fusion protein of about 10 μg/kg to about 20 μg/kg on any one of day 13, day 14, day 15, day 16, or day 17.

In certain embodiments, the patient is administered a first dose of the fusion protein of about 15 μg/kg on day 1, a second dose of the fusion protein of about 15 μg/kg on day 4, and a third dose of the fusion protein of about 15 μg/kg on day 8.

In certain embodiments, the patient is administered a first dose of the fusion protein of about 20 μg/kg on day 1, a second dose of the fusion protein of about 20 μg/kg on day 8, and a third dose of the fusion protein of about 20 μg/kg on day 15.

In certain embodiments, the first dose, the second dose, and the third dose are the same. In certain embodiments, the first dose, the second dose, and the third dose are different.

In certain embodiments, the patient is administered a fourth dose of the fusion protein.

In certain embodiments, the patient is administered the third dose of the fusion protein and the fourth dose of the fusion protein on consecutive days.

In certain embodiments, the patient is administered the third dose of the fusion protein and the fourth dose of the fusion protein on non-consecutive days.

In another aspect, the disclosure provides a method of treating cancer in a patient comprising administering to the patient a fusion protein comprising an amino acid sequence set forth in SEQ ID NO: 1, or a fusion protein comprising at least 80% identity to the amino acid sequence set forth in SEQ ID NO: 1, wherein the patient is administered at least a first dose of the fusion protein, wherein the patient is not administered a dose of the fusion protein at least one day prior to the first dose and the patient is not administered a dose of the fusion protein one day after the first dose.

In another aspect, the disclosure provides a method of treating cancer in a patient comprising administering to the patient a fusion protein comprising an amino acid sequence set forth in SEQ ID NO: 1, or a fusion protein comprising at least 80% identity to the amino acid sequence set forth in SEQ ID NO: 1, wherein the patient is administered a single dose of the fusion protein once every week (Q1W), once every 2 weeks (Q2W), once every 3 weeks (Q3W), or once every 4 weeks (Q4W).

In certain embodiments, the fusion protein is administered at a dose of about 10 μg/kg to about 60 μg/kg.

In certain embodiments, the fusion protein is administered at a dose of about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, or about 60 μg/kg.

In certain embodiments, the fusion protein is administered at a dose of about 10 μg/kg, about 15 μg/kg, about 20 μg/kg, about 25 μg/kg, about 30 μg/kg, about 35 μg/kg, or about 40 μg/kg.

In certain embodiments, administration of the fusion protein results in a dose dependent increase in circulating NK cells and CD8+ cells in the patient in the absence of a dose dependent increase in T regulatory (Treg) cells. In certain embodiments, the increase in circulating NK cells and CD8+ cells is at least 2 fold over baseline. In certain embodiments, the increase in circulating NK cells and CD8+ cells is greater relative to the increase in circulating Treg cells.

In certain embodiments, an increase in circulating NK cells and CD8+ cells is greater relative to the increase in circulating Treg cells as compared to the increase in circulating NK cells and CD8+ cells relative to the increase in circulating Treg cells in a patient receiving high dose rhIL-2 treatment.

In certain embodiments, the patient has an improved safety profile as compared to a patient receiving high dose recombinant human IL-2 (rhIL-2) treatment.

In certain embodiments, the patient has a lower risk of capillary leak syndrome or cytokine release syndrome and/or the patient has a lower risk of cytokine release syndrome as compared to a patient receiving high dose rhIL-2 treatment.

In certain embodiments, the method results in a dose dependent increase in circulating NK cells and CD8+ cells in the patient in the absence of a dose dependent increase in circulating T regulatory (Treg) cells and wherein the increase in circulating NK cells and CD8+ cells relative to the increase in circulating T regulatory (Treg) is greater as compared to the increase in circulating NK cells and CD8+ cells relative to the increase in circulating T regulatory (Treg) in a patient receiving high dose recombinant human IL-2 (rhIL-2) treatment, and wherein the patient has a lower risk of capillary leak syndrome.

In certain embodiments, each dose is administered by intravenous (I.V.) injection or infusion.

In certain embodiments, each dose is administered by intravenous (I.V.) injection or infusion over a period of about 30 minutes, about 60 minutes, or about 90 minutes.

In certain embodiments, the method further comprises administering to the patient a therapeutically effective amount of a therapeutic agent.

In certain embodiments, the therapeutic agent is a PARP inhibitor, an immune checkpoint inhibitor a cytotoxic agent, or a chemotherapeutic agent.

In certain embodiments, the therapeutic agent is an immune checkpoint inhibitor.

In certain embodiments, the immune checkpoint inhibitor inhibits the interaction of PD-1 and PD-L1.

In certain embodiments, the immune checkpoint inhibitor is pembrolizumab.

In certain embodiments, the pembrolizumab is administered prior to, simultaneously with, or subsequent to, administration of the fusion protein of SEQ ID NO: 1.

In certain embodiments, the pembrolizumab is administered in a separate composition from the fusion protein of SEQ ID NO: 1.

In certain embodiments, the pembrolizumab is administered in an amount of 200 mg by I.V. injection or infusion.

In certain embodiments, the pembrolizumab is administered once every week (Q1W), once every 2 weeks (Q2W), once every 3 weeks (Q3W), or once every 4 weeks (Q4W).

In certain embodiments, the pembrolizumab is administered in an amount of 200 mg by I.V. injection or infusion Q3W.

In certain embodiments, the pembrolizumab is administered on the same day as the first dose of the fusion protein.

In certain embodiments, the cancer being treated is a solid tumor.

In certain embodiments, the solid tumor is a carcinoma, sarcoma or lymphoma.

In certain embodiments, the cancer being treated is renal cell carcinoma (RCC), melanoma, mucosal melanoma, breast cancer, pancreatic cancer, prostate cancer, non-small cell lung cancer, liver cancer, colon and rectal cancer, bladder cancer, cervical cancer, ovarian cancer, thyroid cancer, esophageal cancer, oral cancer, mesothelioma, non-melanoma skin cancer, and/or gastric cancer.

In certain embodiments, the cancer being treated is a blood cancer.

In certain embodiments, the blood cancer is leukemia, non-Hodgkin lymphoma, Hodgkin lymphoma and multiple myeloma.

In one aspect, the disclosure provides a pharmaceutical composition comprising a fusion protein comprising an amino acid sequence set forth in SEQ ID NO: 1, or a fusion protein comprising at least 80% identity to the amino acid sequence set forth in SEQ ID NO: 1, wherein the pharmaceutical composition comprises a unit dosage of about 1 μg/kg to about 60 μg/kg.

In certain embodiments, the unit dose is of about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 21 μg/kg, about 22 μg/kg, about 23 μg/kg, about 24 μg/kg, about 25 μg/kg, about 26 μg/kg, about 27 μg/kg, about 28 μg/kg, about 29 μg/kg, about 30 μg/kg, about 31 μg/kg, about 32 μg/kg, about 33 μg/kg, about 34 μg/kg, about 35 μg/kg, about 36 μg/kg, about 37 μg/kg, about 38 μg/kg, about 39 μg/kg, about 40 μg/kg, about 41 μg/kg, about 42 μg/kg, about 43 μg/kg, about 44 μg/kg, about 45 μg/kg, about 46 μg/kg, about 47 μg/kg, about 48 μg/kg, about 49 μg/kg, about 50 μg/kg, about 51 μg/kg, about 52 μg/kg, about 53 μg/kg, about 54 μg/kg, about 55 μg/kg, about 56 μg/kg, about 57 μg/kg, about 58 μg/kg, about 59 μg/kg, or about 60 μg/kg.

In certain embodiments, the unit dose is of about 3 μg/kg, about 6 μg/kg, about 10 μg/kg, about 15 μg/kg, about 20 μg/kg, about 25 μg/kg, about 30 μg/kg, about 35 μg/kg, or about 40 μg/kg.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows a study design schematic for a less-frequent dosing regimen with the fusion protein of SEQ ID NO: 1 (nemvaleukin). Abbreviations: D=day; IV=intravenous; n=number of patients; Q3W-administered every 3 weeks; MTD=maximum tolerated dose.

FIG. 2A-FIG. 2B depict simulations of CD8+ T cells in blood (FIG. 2A) and tumor (FIG. 2B) in a 1 dose/21-day cycle administration scheme. The simulations were run for doses of 15 μg/kg, 23 μg/kg, 30 μg/kg, 37 μg/kg, and 45 μg/kg, and compared against 3 μg/kg and 6 μg/kg with the previously qdx5 administration scheme.

FIG. 3A-FIG. 3B depict simulations of NK cells in blood (FIG. 3A) and tumor (FIG. 3B) in a 1 dose/21-day cycle administration scheme. The simulations were run for doses of 15 μg/kg, 23 μg/kg, 30 μg/kg, 37 μg/kg, and 45 μg/kg, and compared against 3 μg/kg and 6 μg/kg with the previously qdx5 administration scheme.

FIG. 4A-FIG. 4B depict simulations of CD8+ T cells in blood (FIG. 4A) and tumor (FIG. 4B) in a 2 dose, Day 1 and Day 4 dosing/21-day cycle administration scheme. The simulations were run for doses of 7.5 μg/kg, 10 μg/kg, and 15 μg/kg, and compared against 3 μg/kg and 6 μg/kg with the previously qdx5 administration scheme.

FIG. 5A-FIG. 5B depict simulations of NK cells in blood (FIG. 5A) and tumor (FIG. 5B) in a 2 dose, Day 1 and Day 4 dosing/21-day cycle administration scheme. The simulations were run for doses of 7.5 μg/kg, 10 μg/kg, and 15 μg/kg, and compared against 3 μg/kg and 6 μg/kg with the previously qdx5 administration scheme.

FIG. 6A-FIG. 6B depict simulations of CD8+ T cells in blood (FIG. 6A) and tumor (FIG. 6B) in a 2 dose, Day 1 and Day 8 dosing/21-day cycle administration scheme. The simulations were run for doses of 7.5 μg/kg, 10 μg/kg, 15 μg/kg, and 15 μg/kg, and compared against 3 μg/kg and 6 μg/kg with the previously qdx5 administration scheme.

FIG. 7A-FIG. 7B depict simulations NK cells in blood (FIG. 7A) and tumor (FIG. 7B) in a 2 dose, Day 1 and Day 8 dosing/21-day cycle administration scheme. The simulations were run for doses of 7.5 μg/kg, 10 μg/kg, 15 μg/kg, and 15 μg/kg, and compared against 3 μg/kg and 6 μg/kg with the previously qdx5 administration scheme.

FIG. 8A-FIG. 8B depict simulations of CD8+ T cells in blood (FIG. 8A) and tumor (FIG. 8B) in a 3 dose, Day 1, Day 4, Day 8 dosing/21-day cycle administration scheme. The simulations were run for doses of 5 μg/kg, 7.5 μg/kg, and 10 μg/kg, and compared against 3 μg/kg and 6 μg/kg with the previously qdx5 administration scheme.

FIG. 9A-FIG. 9B depict simulations of NK cells in blood (FIG. 9A) and tumor (FIG. 9B) in a 3 dose, Day 1, Day 4, Day 8 dosing/21-day cycle administration scheme. The simulations were run for doses of 5 μg/kg, 7.5 μg/kg, and 10 μg/kg, and compared against 3 μg/kg and 6 μg/kg with the previously qdx5 administration scheme.

FIG. 10A-FIG. 10B depict simulations of CD8+ T cells in blood (FIG. 10A) and tumor (FIG. 10B) in a 3 dose, Day 1, Day 8, Day 15 dosing/21-day cycle administration scheme. The simulations were run for doses of 5 μg/kg, 7.5 μg/kg, 10 μg/kg, and 15 μg/kg, and compared against 3 μg/kg and 6 μg/kg with the previously qdx5 administration scheme.

FIG. 11A-FIG. 11B depict simulations of NK cells in blood (FIG. 11A) and tumor (FIG. 11B) in a 3 dose, Day 1, Day 8, Day 15 dosing/21-day cycle administration scheme. The simulations were run for doses of 5 μg/kg, 7.5 μg/kg, 10 μg/kg, and 15 μg/kg, and compared against 3 μg/kg and 6 μg/kg with the previously qdx5 administration scheme.

### DETAILED DESCRIPTION

The dosing regimens described herein are designed for less frequent dosing, i.e., dosing of the fusion protein of SEQ ID NO: 1 on non-consecutive days. These dosing regimens have the benefit of being easy to administer for patients and healthcare professionals without a substantial decrease in safety and efficacy. These dosing regimens represent an improvement over the existing dosing regimen of the fusion protein of SEQ ID NO: 1, which requires administration for 5 consecutive days (qdx5) for each 21-day cycle of treatment (i.e., Q3W cycle).

Thus, in one aspect the disclosure provides a method of treating cancer in a patient comprising administering to the patient a fusion protein comprising an amino acid sequence set forth in SEQ ID NO: 1, or a fusion protein comprising at least 80% identity to the amino acid sequence set forth in SEQ ID NO: 1, wherein the patient is administered at least a first dose of the fusion protein and a second dose of the fusion protein on non-consecutive days.

In another aspect, the disclosure provides a method of treating cancer in a patient comprising administering to the patient a fusion protein comprising an amino acid sequence set forth in SEQ ID NO: 1, or a fusion protein comprising at least 80% identity to the amino acid sequence set forth in SEQ ID NO: 1, wherein the patient is administered at least a first dose of the fusion protein, wherein the patient is not administered a dose of the fusion protein at least one day prior to the first dose and the patient is not administered a dose of the fusion protein one day after the first dose.

### Definitions

Those skilled in the art will recognize or be able to ascertain using no more than routine experimentation, many equivalents to the specific embodiments in accordance with the invention described herein. The scope of the present invention is not intended to be limited to the following description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

As used herein, the term “about” or “approximately” as applied to one or more values of interest, refers to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein any form of administration or coadministration of a “combination”, “combined therapy” and/or “combined treatment regimen” refers to at least two therapeutically active agents or compositions which may be administered or co-administered”, simultaneously, in either separate or combined formulations, or sequentially at different times separated by minutes, hours or days. In general, each agent will be administered at a dose and/or on a time schedule determined for that agent.

As used herein, the term “parenteral” refers to dosage forms that are intended for administration as an injection or infusion and includes subcutaneous, intravenous, intra-arterial, intraperitoneal, intracardiac, intrathecal, and intramuscular injection, as well as infusion injections usually by the intravenous route.

The term “therapeutic agent” encompasses any agent administered to treat a symptom or disease in an individual in need of such treatment in addition to, or in combination with, the fusion protein of SEQ ID NO: 1. Such additional therapeutic agent may comprise any active ingredients suitable for the particular indication being treated, preferably those with complementary activities that do not adversely affect each other.

The term “chemotherapeutic agent” refers to a compound or a derivative thereof that can interact with a cancer cell, thereby reducing the proliferative status of the cell and/or killing the cell for example, by impairing cell division or DNA synthesis, or by damaging DNA, effectively targeting fast dividing cells. Examples of chemotherapeutic agents include, but are not limited to, alkylating agents (e.g., cyclophosphamide, ifosfamide); metabolic antagonists (e.g., methotrexate (MTX), 5-fluorouracil or derivatives thereof); a substituted nucleotide; a substituted nucleoside; DNA demethylating agents (also known as antimetabolites; e.g., azacitidine); antitumor antibiotics (e.g., mitomycin, adriamycin); plant-derived antitumor agents (e.g., vincristine, vindesine, TAXOL®, paclitaxel, abraxane); cisplatin; carboplatin; etoposide; and the like. Such agents may further include, but are not limited to, the anti-cancer agents trimethotrexate (TMTX); temozolomide; raltitrexed; S-(4-Nitrobenzyl)-6-thioinosine (NBMPR); 6-benzyguanidine (6-BG); a nitrosoureas a nitrosourea (rabinopyranosyl-N-methyl-N-nitrosourea (Aranose), Carmustine (BCNU, BICNU), Chlorozotocin, Ethylnitrosourea (ENU), Fotemustine, Lomustine (CCNU), Nimustine, N-Nitroso-N-methylurea (NMU), Ranimustine (MCNU), Semustine, Streptozocin (Streptozotocin)); cytarabine; and camptothecin; or a therapeutic derivative of any thereof.

As used herein a single “course” of treatment such as a first course, second course, third course and so on refers to a treatment regimen wherein the Fusion Protein is administered for a desired period of time such as 2 or 3 doses on non-consecutive days of treatment followed by a rest period of a certain amount of consecutive days.

The term “fusion protein” designates a protein or peptide linked together with another protein or peptide by peptide bond between their respective N- and C-terminal amino acid residues or verse visa, or by insertion of the first protein or peptide into the internal region of the second protein or peptide by two peptide bonds at the N- and C-termini of the inserted protein or peptide. A peptide bond is a covalent chemical bond formed between carboxyl group of one amino acid and the amine group of another amino acid. A fusion protein is produced by expression of the fusion protein gene in an expression host, in which the coding sequence for the first protein or peptide is linked to the coding sequence of the second protein or peptide.

The disclosure also contemplates the use of a variant of the fusion protein of SEQ ID NO: 1 having an amino acid sequence having sequence identity that is about 75%, 80%, 85%, 90%, 91%, 92%, 93%, 94%, 95%, 96%, 97%, 98%, 99% or higher over a contiguous stretch of about 20 amino acids up to the full length of SEQ ID NO: 1. A variant of the SEQ ID NO: 1 may have a defined sequence identity as compared to SEQ ID NO: 1 over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)).

As an example, a variant of the fusion protein of SEQ ID NO: 1 can comprise an amino acid sequence having at least about 75%, at least about 80%, at least about 85%, at least about 90%, at least about 95%, at least about 98%, or at least about 99%, amino acid sequence identity to a contiguous stretch of SEQ ID NO: 1 of at least 20 amino acids and preferably from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length of SEQ ID NO: 1.

The term “IL-2 therapy” includes administration of immunotherapy based on IL-2 and its associated biological functions as an immunotherapy including but not limited to maintenance of CD4⁺ regulatory T cells and differentiation of CD4⁺ T cells into a variety of subsets; promotion of CD8⁺ T-cell and NK cell cytotoxicity activity, and modulation of T-cell differentiation programs in response to antigen, promoting naive CD4⁺ T-cell differentiation into T helper-1 (Th1) and T helper-2 (Th2) cells while inhibiting T helper-17 (Th17) differentiation. Therefore “IL-2 therapy” as used herein includes but is not limited to immunotherapy with rhIL-2 or a variant of rhIL-2 such as the Fusion Protein of SEQ ID NO: 1.

The terms “high dose IL-2” and “HD IL-2” include a dose of interleukin-2 (IL-2) of about or at least about 600,000 International Units (IU)/kg of body weight (kg)/dose, or about or at least about 720,000 IU/kg/dose.

The terms “low dose IL-2” and “LD IL-2” include a dose of interleukin-2 (IL-2) of less than about 600,000 IU/kg of body weight/dose, such as about 60,000 or about 72,000 IU/kg/dose, e.g., from about 60,000 to about 72,000 IU/kg/dose.

As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants. Preferably “patient” refers to a human subject who may seek or be in need of treatment, requires treatment, is receiving treatment, will receive treatment, or a subject who is under care by a trained professional for a particular disease or condition. The “patient” can be a child (>1-17 years). In still other embodiments, the patient can be an infant (1 year and younger). In yet still other embodiments, the patient can be a pediatric patient, wherein the term “pediatric” is used as understood by those skilled in the art. For example, pediatric patients include infants, children and adolescents.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “protein” or “peptide” as used herein refers to a at least two or more amino acid residues linked together by peptide bond. The amino acid sequence in a protein or peptide is shown in the standard format, i.e., from amino terminus (N-terminus) to carboxyl terminus (C-terminus).

The term “recombinant production” refers to the techniques for manipulating and combining two or more DNA sequences together that include recombination, PCR (polymerase chain reaction), in vitro mutagenesis, and direct DNA synthesis. These techniques are described in numerous published books and manuals, including the “Current protocols in molecular biology” (Ausubel eds. 2008. John Wiley & Son).

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The phrase “therapeutically effective amount” or an “effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the amount of inflammatory cytokines produced following administration can be indicative of whether a therapeutically effective amount has been used. In reference to cancer or pathologies related to unregulated cell division, a therapeutically effective amount refers to that amount which has the effect of (1) reducing the size of a tumor (i.e. tumor regression), (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer. An “effective amount” is also that amount that results in desirable PD and PK profiles and desirable immune cell profiling upon administration of the therapeutically active compositions of the invention.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease. The term “prevent,” “preventing,” or “prevention” as used herein, comprises the prevention of at least one symptom associated with or caused by the state, disease or disorder being prevented.

“Progression free survival (PFS),” as used in the context of the cancers described herein, refers to the length of time during and after treatment of the cancer until objective tumor progression or death of the patient. The treatment may be assessed by objective or subjective parameters; including the results of a physical examination, neurological examination, or psychiatric evaluation. In preferred aspects, PFS may be assessed by investigator or independent central review.

“Overall survival (OS)” may be assessed by OS rate at certain time points (e.g., 1 year and 2 years) by the Kaplan-Meier method and corresponding 95% CI will be derived based on Greenwood formula by study treatment for each tumor type. OS rate is defined as the proportion of participants who are alive at the time point. OS for a participant is defined as the time from the first dosing date to the date of death due to any cause.

As used herein a “complete response” is either the disappearance of all signs of cancer in response to treatment or that the target lesions measured are <5 mm or <10 mm.

As used herein the term “partial response” means a decrease in the size of the tumor, or in the extent of cancer in the body in response to treatment. A partial response may also be referred to herein as “partial remission”.

The term “cancer”, as used herein, shall be given its ordinary meaning, as a general term for diseases in which abnormal cells divide without control.

The term “reducing a tumor” or “tumor regression” as used herein refers to a reduction in the size or volume of a tumor mass, a decrease in the number of metastasized tumors in a subject, a decrease in the proliferative status (the degree to which the cancer cells are multiplying) of the cancer cells, and the like.

The term “enhancing”, as used herein, refers to allowing a subject or tumor cell to improve its ability to respond to a treatment disclosed herein. For example, an enhanced response may comprise an increase in responsiveness of at least 5%, 10%, 15%, 20%, 25%, 30%, 35%, 40%, 45%, 50%, 55%, 60%, 65%, 70%, 75%, 80%, 85%, 90%, 95% or 98% or more. As used herein, “enhancing” can also refer to enhancing the number of subjects who respond to a treatment such as a combination therapy comprising chemotherapy, drug-resistant immunocompetent cells, and immune checkpoint inhibitors. For example, an enhanced response may refer to a total percentage of subjects who respond to a treatment wherein the percentage is of at least 5%, 10%, 15%, 20%, 25%, 30%, 35%, 40%, 45%, 50%, 55%, 60%, 65%, 70%, 75%, 80%, 85%, 90%, 95% or 98% or more.

“Immune checkpoint proteins” regulate T cell function in the immune system. T cells play a central role in cell-mediated immunity. Immune checkpoint proteins interact with specific ligands that send a signal into the T cell and essentially switch off or inhibit T cell function. Cancer cells take advantage of this system by driving high levels of expression of immune checkpoint proteins on their surface that results in control of the T cells expressing immune checkpoint proteins on the surface of T cells that enter the tumor microenvironment, thus suppressing the anticancer immune response. As such, inhibition of immune checkpoint proteins by agents referred to herein as “immune checkpoint protein inhibitors” or “immune checkpoint inhibitors” would result in restoration of T cell function and an immune response to the cancer cells. Examples of immune checkpoint proteins include, but are not limited to: CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, OX40, B-7 family ligands or a combination thereof. In certain embodiments, the immune checkpoint inhibitor interacts with a ligand of an immune checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, OX40, A2aR, B-7 family ligands or a combination thereof. Examples of immune checkpoint inhibitors include but are not limited to: a PD-1 antagonist, a PD-L1 antagonist, a CTLA-4 antagonist, an adenosine A2A receptor antagonist, a B7-H3 antagonist, a B7-H4 antagonist, a BTLA antagonist, a KIR antagonist, a LAG3 antagonist, a TIM-3 antagonist, a VISTA antagonist, or a TIGIT antagonist.

As used herein the term “angiogenesis inhibitor” refers to a drug, compound, antibody or other agent that keeps new blood vessels from forming. In cancer treatment, angiogenesis inhibitors may prevent the growth of new blood vessels that tumors need to grow. Angiogenesis inhibitors include those agents that can target one or more signaling pathways associated with receptor tyrosine kinases (RTK). RTKs include, but are not limited to, vascular endothelial growth factor receptors types 1, 2, and 3 (VEGFR1-3); platelet derived growth factor receptors, types alpha and beta (PDGFRα/β) and fibroblast growth factor receptors (FGFR), types 1, 2, and 3 (FGFR1-3). In certain embodiments, the angiogenesis inhibitors have broad target selectivity and are capable of simultaneous targeted inhibition of multiple RTKs and are referred to herein as “multiple receptor tyrosine kinase inhibitors”.

### Fusion Protein of SEQ ID NO: 1

A recombinant human IL-2 variant fusion protein, described in WO 2013/184942, is a circularly permuted (cp) IL-2 variant fused to the extracellular domain of the IL-2Rα portion of the IL-2 receptor and is referred to herein as the “fusion protein of SEQ ID NO: 1” or “nemvaleukin” or “nemvaleukin alfa” and has the following amino acid sequence:

(SEQ ID NO: 1)
SKNFHLRPRDLISNINVIVLELKGSETTFMCEYADETATIVEFLNRWITF
SQSIISTLTGGSSSTKKTQLQLEHLLLDLQMILNGINNYKNPKLTRMLTF
KFYMPKKATELKHLQCLEEELKPLEEVLNLAQGSGGGSELCDDDPPEIPH
ATFKAMAYKEGTMLNCECKRGFRRIKSGSLYMLCTGNSSHSSWDNQCQCT
SSATRNTTKQVTPQPEEQKERKTTEMQSPMQPVDQASLPGHCREPPPWEN
EATERIYHFVVGQMVYYQCVQGYRALHRGPAESVCKMTHGKTRWTQPQLI
CTG.

It is contemplated that fusion proteins that are closely related to SEQ ID NO: 1, such as those fusion proteins having sequence identities of about 80%, 85%, 90%, 91%, 92%, 93%, 94%, 95%, 96%, 97%, 98%, 99% or higher identity over the full length of SEQ ID NO: 1 may also be suitable for administration in accordance with the methods of the invention. It is contemplated that fusion proteins that are closely related to SEQ ID NO: 1, such as those fusion proteins having sequence identities of about 80%, 85%, 90%, 91%, 92%, 93%, 94%, 95%, 96%, 97%, 98%, 99% or higher identity over a contiguous sequence of at least about 20 amino acids up to the full length of SEQ ID NO: 1 may also be suitable for administration in accordance with the methods of the invention.

The fusion protein of SEQ ID NO: 1 may be produced using a biological recombinant expression system or any protein synthesizer. Strategies for recombinant protein expression are well known in the art, and typically involve transfecting cells with a DNA vector that contains a genetic template encoding the Fusion Protein of SEQ ID NO: 1 and then culturing the cells so that they transcribe and translate the Fusion Protein. Typically, the cells are then lysed to extract the expressed protein for subsequent purification. Both prokaryotic and eukaryotic in vivo protein expression systems are widely used. Preferably, the fusion protein of SEQ ID NO: 1 is produced in CHO cells.

The invention provides pharmaceutical compositions of a dose of at least about 1 μg/kg to about 60 μg/kg of the fusion protein of SEQ ID NO: 1.

A pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof.

Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this present disclosure.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use. General considerations in the formulation and/or manufacture of pharmaceutical agents may be found, for example, in Remington's The Science and Practice of Pharmacy, 21^{st} Edition, A. R. Gennaro (Lippincott, Williams & Wilkins, Baltimore, Md., 2006; incorporated herein by reference).

The fusion protein of SEQ ID NO: 1 is designed to selectively bind to and activate the intermediate-affinity IL-2R, but not the high-affinity IL-2R. The IL-2Rα domain of the fusion protein of SEQ ID NO: 1 serves to sterically impede the binding of the fusion protein of SEQ ID NO: 1 to the high-affinity IL-2R yet still allow binding to the intermediate-affinity IL-2R.

In vitro and in vivo nonclinical pharmacodynamic (PD) data support selective signaling through the intermediate-affinity IL-2 receptor by the fusion protein of SEQ ID NO: 1, leading to the activation and expansion of effector cells such as NK cells and CD8+ cells, while minimizing the activation and expansion of immunosuppressive T_{regs}. Additionally, in vivo in mice, the fusion protein of SEQ ID NO: 1 displays improved tolerability relative to rhIL-2 at doses that elicit equivalent or greater expansion of effector cells relative to T_{regs}.

Additional nonclinical data demonstrate that IV or SC administration of the fusion protein of SEQ ID NO: 1 results in equivalent tumor growth inhibition in a mouse syngeneic tumor model, as well as similar peripheral expansion of NK and CD8⁺ T cells after either route of administration to cynomolgus monkeys.

First in human clinical data described in Example 1, indicates that the fusion protein of SEQ ID NO: 1 activates expansion of CD8+ cells and NK cells in a dose dependent manner in the absence of dose dependent activation of Tregs. Therefore, the fusion protein of SEQ ID NO: 1 can be dosed in human patients at a concentration that is comparative to high dose rhIL-2 to elicit equivalent or greater expansion of NK cells and CD8+ cells as compared to high dose rhIL-2 but with far less (at least two-fold less) relative expansion of immunosuppressive Tregs as compared to high dose rhIL-2. This result was unexpected.

### Dosing Regimens

The fusion protein of SEQ ID NO: 1 is administered to a cancer patient in accordance with the methods and dosing regimens of the disclosure. In certain embodiments, the administration route is intravenous, e.g., intravenous injection and intravenous infusion, e.g., via central venous access. Additional routes of administration include subcutaneous, intramuscular, oral, nasal, and pulmonary administration. In certain embodiments, the fusion protein of SEQ ID NO: 1 may be administered as part of a pharmaceutical composition comprising at least one excipient. The dosing regimens described herein are designed for less frequent dosing, i.e., dosing of the fusion protein of SEQ ID NO: 1 on non-consecutive days. These dosing regimens have the benefit of being easy to administer for patients and healthcare professionals without a substantial decrease in safety and efficacy. These dosing regimens represent an improvement over the existing dosing regimen of the fusion protein of SEQ ID NO: 1, which requires administration for 5 consecutive days (qdx5) for each 21-day cycle of treatment (i.e., Q3W cycle).

In one aspect, the disclosure provides a method of treating cancer in a patient comprising administering to the patient a fusion protein comprising an amino acid sequence set forth in SEQ ID NO: 1, or a fusion protein comprising at least 80% identity to the amino acid sequence set forth in SEQ ID NO: 1, wherein the patient is administered at least a first dose of the fusion protein and a second dose of the fusion protein on non-consecutive days.

In another aspect, the disclosure provides a method of treating cancer in a patient comprising administering to the patient a fusion protein comprising an amino acid sequence set forth in SEQ ID NO: 1, or a fusion protein comprising at least 80% identity to the amino acid sequence set forth in SEQ ID NO: 1, wherein the patient is administered at least a first dose of the fusion protein, wherein the patient is not administered a dose of the fusion protein at least one day prior to the first dose and the patient is not administered a dose of the fusion protein one day after the first dose.

In certain embodiments, the fusion protein is administered at a dose of about 1 μg/kg to about 60 μg/kg.

In certain embodiments, the fusion protein is administered at a dose of about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 21 μg/kg, about 22 μg/kg, about 23 μg/kg, about 24 μg/kg, about 25 μg/kg, about 26 μg/kg, about 27 μg/kg, about 28 μg/kg, about 29 μg/kg, about 30 μg/kg, about 31 μg/kg, about 32 μg/kg, about 33 μg/kg, about 34 μg/kg, about 35 μg/kg, about 36 μg/kg, about 37 μg/kg, about 38 μg/kg, about 39 μg/kg, about 40 μg/kg, about 41 μg/kg, about 42 μg/kg, about 43 μg/kg, about 44 μg/kg, about 45 μg/kg, about 46 μg/kg, about 47 μg/kg, about 48 μg/kg, about 49 μg/kg, about 50 μg/kg, about 51 μg/kg, about 52 μg/kg, about 53 μg/kg, about 54 μg/kg, about 55 μg/kg, about 56 μg/kg, about 57 μg/kg, about 58 μg/kg, about 59 μg/kg, or about 60 μg/kg.

In certain embodiments, the fusion protein is administered at a dose of about 3 μg/kg, about 6 μg/kg, about 10 μg/kg, about 15 μg/kg, about 20 μg/kg, about 25 μg/kg, about 30 μg/kg, about 35 μg/kg, or about 40 μg/kg.

In certain embodiments, the first dose and the second dose are the same (i.e., a first dose of about 10 μg/kg and a second dose of about 10 μg/kg). In other embodiments, the first dose and the second dose are different (i.e., a first dose of about 15 μg/kg and a second dose of about 10 μg/kg).

In certain embodiments, the patient is administered a third dose of the fusion protein.

In certain embodiments, the patient is administered the first dose of the fusion protein on day 1 and the second dose of the fusion protein on any one of day 3, day 4, day 5, day 6, day 7, day 8, day 9, day 10, day 11, day 12, day 13, day 14, day 15, day 16, day 17, day 18, day 19, day 20, or day 21.

In certain embodiments, the patient is administered the first dose of the fusion protein on day 1 and the second dose of the fusion protein on day 4 (i.e., a 1-4 dosing scheme).

In certain embodiments, the patient is administered the first dose of the fusion protein on day 1 and the second dose of the fusion protein on day 8 (i.e., a 1-8 dosing scheme).

In certain embodiments, the patient is administered the first dose of the fusion protein on day 1 and the second dose of the fusion protein on day 15 (i.e., a 1-15 dosing scheme).

In certain embodiments, the patient is administered the second dose of the fusion protein and the third dose of the fusion protein on consecutive days (i.e., second dose on day 4 and third dose on day 5). In other embodiments, the patient is administered the second dose of the fusion protein and the third dose of the fusion protein on non-consecutive days (i.e., second dose on day 4 and third dose on day 8). In certain embodiments, the patient is administered the third dose of the fusion protein 1 day, 2 days, 3 days, 4 days, 5 days, 6 days, 7 days, 8 days, 9 days, or 10 days after the second dose of the fusion protein.

In certain embodiments, the patient is administered the first dose of the fusion protein on day 1, the second dose of the fusion protein on any one of day 3, day 4, day 5, day 6, day 7, day 8, day 9, day 10, day 11, or day 12, and the third dose of the fusion protein on any one of day 13, day 14, day 15, day 16, day 17, day 18, day 19, day 20, or day 21.

In certain embodiments, the patient is administered the first dose of the fusion protein on day 1, the second dose of the fusion protein on any one of day 3, day 4, or day 5, and the third dose of the fusion protein on any one of day 6, day 7, day 8, day 9, or day 10.

In certain embodiments, the patient is administered the first dose of the fusion protein on day 1, the second dose of the fusion protein on any one of day 6, day 7, day 8, day 9, or day 10, and the third dose of the fusion protein on any one of day 13, day 14, day 15, day 16, or day 17.

In certain embodiments, the patient is administered the first dose of the fusion protein on day 1, the second dose of the fusion protein on day 4, and the third dose of the fusion protein on day 8 (i.e., a 1-4-8 dosing scheme).

In certain embodiments, the patient is administered the first dose of the fusion protein on day 1, the second dose of the fusion protein on day 8, and the third dose of the fusion protein on day 15 (i.e., a Jan. 8, 2015 dosing scheme).

In certain embodiments, the patient is administered a first dose of the fusion protein of about 10 μg/kg to about 30 μg/kg on day 1, and a second dose of the fusion protein of about 10 μg/kg to about 30 μg/kg on any one of day 3, day 4, day 5, day 6, day 7, day 8, day 9, day 10, day 11, day 12, day 13, day 14, day 15, day 16, day 17, day 18, day 19, day 20, or day 21.

In certain embodiments, the patient is administered a first dose of the fusion protein of about 10 μg/kg to about 15 μg/kg on day 1, and a second dose of the fusion protein of about 10 μg/kg to about 15 μg/kg on any one of day 3, day 4, day 5, or day 6.

In certain embodiments, the patient is administered a first dose of the fusion protein of about 10 μg/kg to about 25 μg/kg on day 1, and a second dose of the fusion protein of about 20 μg/kg to about 25 μg/kg on any one of day 6, day 7, day 8, day 9, or day 10.

In certain embodiments, the patient is administered a first dose of the fusion protein of about 15 μg/kg on day 1, and a second dose of the fusion protein of about 15 μg/kg on day 4.

In certain embodiments, the patient is administered a first dose of the fusion protein of about 20 μg/kg to about 25 μg/kg on day 1, and a second dose of the fusion protein of about 20 μg/kg to about 25 μg/kg on day 8.

In certain embodiments, the patient is administered a first dose of the fusion protein of about 6 μg/kg to about 25 μg/kg on day 1, a second dose of the fusion protein of about 6 μg/kg to about 25 μg/kg on any one of day 3, day 4, day 5, day 6, day 7, day 8, day 9, day 10, day 11, or day 12, and a third dose of the fusion protein of about 6 μg/kg to about 25 μg/kg on any one of day 13, day 14, day 15, day 16, day 17, day 18, day 19, day 20, or day 21.

In certain embodiments, the patient is administered a first dose of the fusion protein of about 6 μg/kg to about 15 μg/kg on day 1, a second dose of the fusion protein of about 6 μg/kg to about 15 μg/kg on any one of day 3, day 4, or day 5, and a third dose of the fusion protein of about 6 μg/kg to about 15 μg/kg on any one of day 6, day 7, day 8, day 9, or day 10.

In certain embodiments, the patient is administered a first dose of the fusion protein of about 10 μg/kg to about 20 μg/kg on day 1, a second dose of the fusion protein of about 10 μg/kg to about 20 μg/kg on any one of day 6, day 7, day 8, day 9, or day 10, and a third dose of the fusion protein of about 10 μg/kg to about 20 μg/kg on any one of day 13, day 14, day 15, day 16, or day 17.

In certain embodiments, the patient is administered a first dose of the fusion protein of about 15 μg/kg on day 1, a second dose of the fusion protein of about 15 μg/kg on day 4, and a third dose of the fusion protein of about 15 μg/kg on day 8.

In certain embodiments, the patient is administered a first dose of the fusion protein of about 20 μg/kg on day 1, a second dose of the fusion protein of about 20 μg/kg on day 8, and a third dose of the fusion protein of about 20 μg/kg on day 15.

In certain embodiments, the first dose, the second dose, and the third dose are the same (i.e., a first dose of about 10 μg/kg, a second dose of about 10 μg/kg, and a third dose of about 10 μg/kg). In other embodiments, the first dose, the second dose, and the third dose are different (i.e., a first dose of about 20 μg/kg, a second dose of about 15 μg/kg, and a third dose of about 10 μg/kg).

In certain embodiments, the patient is administered a fourth dose of the fusion protein. In certain embodiments, the patient is administered the third dose of the fusion protein and the fourth dose of the fusion protein on consecutive days. In certain embodiments, the patient is administered the third dose of the fusion protein and the fourth dose of the fusion protein on non-consecutive days.

In yet another aspect, the disclosure provides a method of treating cancer in a patient comprising administering to the patient a fusion protein comprising an amino acid sequence set forth in SEQ ID NO: 1, or a fusion protein comprising at least 80% identity to the amino acid sequence set forth in SEQ ID NO: 1, wherein the patient is administered a single dose of the fusion protein once every week (Q1W), once every 2 weeks (Q2W), once every 3 weeks (Q3W), or once every 4 weeks (Q4W).

In certain embodiments, the fusion protein is administered at a dose of about 10 μg/kg to about 60 μg/kg. In certain embodiments, the fusion protein is administered at a dose of about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, or about 60 μg/kg. In certain embodiments, the fusion protein is administered at a dose of about 10 μg/kg, about 15 μg/kg, about 20 μg/kg, about 25 μg/kg, about 30 μg/kg, about 35 μg/kg, or about 40 g/kg.

In certain embodiments, the fusion protein of SEQ ID NO: 1 is administered as a single I.V. infusion each day. A single I.V. infusion may take from 5 minutes to 2 hours. In certain embodiments, each dose is administered by intravenous (I.V.) injection or infusion over a period of about 30 minutes, about 60 minutes, or about 90 minutes

In certain embodiments, the dosing regimen for administration of the fusion protein of SEQ ID NO: 1 provides for one or more treatment courses (i.e., treatment cycles). A single treatment course may take place over a period of days ranging from 1-90 days. Preferably a single treatment course extends for a period of 14 days or 21 days.

In certain embodiments, the first course of treatment comprises administering the fusion protein of SEQ ID NO: 1 by I.V. infusion on non-consecutive days followed by a rest period of a select number of days for a first treatment course that lasts about 21 days (i.e., 21 days counting from day 1 when the first dose of the fusion protein of SEQ ID NO: 1 is administered). Generally, a second course of treatment follows the first course of treatment. The second course of treatment may begin at any time after the first course of treatment but preferably begins within about 24 hours or more after the first course of treatment has ended.

In certain embodiments, the fusion protein of SEQ ID NO: 1 is administered with another therapeutic and/or anti-cancer agent as described infra. Preferably the therapeutic agent is the immune checkpoint inhibitor, pembrolizumab. Preferably pembrolizumab is administered in a separate composition from the fusion protein of SEQ ID NO: 1 and is preferably administered by I.V. infusion prior to, subsequent to, or simultaneously infusion of the fusion protein of SEQ ID NO: 1. In certain embodiments, pembrolizumab is administered at a dose of about 200 mg every two weeks (Q2W), every three weeks (Q3W), every four weeks (Q4W), or as per the standard prescribing recommendations. In certain embodiments, pembrolizumab is administered on the first day of each course of treatment with the fusion protein of SEQ ID NO: 1. Preferably, when co-administering the Fusion Protein with pembrolizumab, the first course of treatment with the fusion protein and all subsequent courses of treatment (e.g., Treatment courses 2, 3, 4 and 5) are generally about 21-day courses.

All of the dosing regimens of the invention described above preferably result in a dose dependent increase in circulating NK cells and CD8+ cells in a patient in the absence of a dose dependent increase in T regulatory (Treg) cells and preferably result in an increase in circulating NK cells and CD8+ cells that is greater relative to the increase in circulating Treg cells in the patient. As compared to high dose or low dose rhIL-2 therapy, all dosing regimens of the invention preferably require less frequent dosing e.g., once daily dosing of the fusion protein of SEQ ID NO: 1 as compared to dosing 3 times per day dosing of high dose or low dose rhIL-2.

Preferably the increase in circulating CD8+ T-cells resulting from administration of the fusion protein of SEQ ID NO: 1 is at least about a 2-fold, at least about a 3-fold, at least about a 4-fold, at least about a 5-fold, at least about a 6-fold, at least about a 7-fold, at least about an 8-fold, about a 9-fold, about a 10-fold, or more as compared to baseline. Preferably the ratio of increase in circulating CD8+ T cells resulting from administration of the fusion protein of SEQ ID NO: 1 is greater relative to the ratio of increase in circulating T regulatory cells.

Preferably the fusion protein of SEQ ID NO: 1 and pharmaceutical compositions thereof, in combination with one or more immune checkpoint inhibitors to treat and/or prevent various diseases, disorders and conditions (e.g., cancers) is affected by utilizing particular dosing parameters that serve to minimize any adverse effects associated with administration of the individual therapies by themselves. By way of example, the addition of the administration of the fusion protein of SEQ ID NO: 1 in a treatment regimen comprising an immune checkpoint inhibitor (e.g. pembrolizumab) might allow a reduction of the amount of an immune checkpoint inhibitor needed to achieve the therapeutic goal, thus reducing (or even eliminating) severe and fatal immune-mediated adverse reactions that prompted the FDA to require a “black box” warning on certain immune checkpoint inhibitors (e.g. pembrolizumab).

In general, dosing parameters of monotherapy with the fusion protein of SEQ ID NO: 1 or any of the combination therapies described herein dictate that the dosage amount be less than an amount that could be irreversibly toxic to the subject (i.e., the maximum tolerated dose, “MTD”) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount can be more than the calculated ED50, in other situations the effective amount can be less than the calculated ED50, and in still other situations the effective amount can be the same as the calculated ED50.

In addition, an effective dose of the fusion protein of SEQ ID NO: 1 can be an amount that, when administered in one or more doses to a subject, produces a desired result relative to a healthy subject. For example, for a subject experiencing a particular disorder, an effective dose can be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

Preferably, the patient is administered the fusion protein of SEQ ID NO: 1 again if after initial treatment the cancer reoccurs. For example, if the patient is initially treated for a solid tumor, and the tumor returns or more tumors develop, the patient is administered SEQ ID NO: 1, as, for example, another course or series of courses of SEQ ID NO: 1.

Preferably, the fusion protein of SEQ ID NO: 1 is administered to a cancer patient in accordance with the methods and dosing regimens of the invention. Preferred routes of administration are intravenous, e.g., intravenous injection and intravenous infusion, e.g., via central venous access. Additional preferred routes of administration include subcutaneous, intramuscular, oral, nasal, and pulmonary administration.

The treatment regimens of the invention are administered to the patient until the patient is cured or until the patient is no longer benefiting from the treatment regimen.

### Improved Safety Profile

The toxicity of rhIL-2 in humans and animals is well documented. At the high doses of rhIL-2 used in most cancer trials, considerable toxicity has been documented with only occasional tumor responses. One major dose-limiting toxicity of human recombinant interleukin-2 (rhIL-2) is capillary leak syndrome (CLS) also referred to herein as vascular leak syndrome (VLS). CLS is characterized by an increase in vascular permeability accompanied by extravasation of fluids and proteins resulting in interstitial edema and organ failure. Manifestations of CLS include fluid retention, increase in body weight, peripheral edema, pleural and pericardial effusions, ascites, anasarca and, in severe form, signs of pulmonary and cardiovascular failure. Symptoms are highly variable among patients and the causes are poorly understood.

The pathogenesis of endothelial cell (EC) damage is complex and can involve activation or damage to ECs and leukocytes, release of cytokines and of inflammatory mediators, alteration in cell-cell and cell-matrix adhesion and in cytoskeleton function. CLS restricts the doses of IL-2 which can be administered to humans and, in some cases, necessitates the cessation of therapy.

The methods of the invention reduce the risk of side effects often associated with high dose therapy while maintaining the desired therapeutic activity of IL-2 therapy including, but not limited to, CLS as well as cytokine release syndrome (CRS), another syndrome associated with immune therapy with cytokines that often accompanies and/or overlaps with CLS.

Dose escalation studies in human clinical trials as described in the Examples have surprisingly revealed that the administration of the Fusion Polypeptide of SEQ ID NO: 1 to a patient at concentrations that were equivalent to those of high dose rh-IL-2 did not result in frequency and severity of certain side effects, for example, capillary leak syndrome, often associated with high dose rhIL-2 therapy. As described in the Examples outlining human clinical studies, dose limiting toxicities (DLTs) have not yet been reached doses of 6 μg/kg/day and higher even though the EC50 values for natural killer cell and CD8+ T cell activation have been exceeded. Therefore, the methods of the invention may result in an improved safety profile for patients having cancer and in need of IL-2 therapy as compared to, for example, standard rhIL-2 therapy and particularly as compared to high dose rhIL-2 therapy.

As used herein, an “improved safety profile”, or a “lower risk of a side effects”, or “reduced frequency or severity of a side effect” associated with, for example standard rhIL-2 therapy and particularly as compared to high dose rhIL-2 therapy can be assessed in several ways. A side effect or symptom of IL-2 therapy may be quantified. A side effect or symptom of IL-2 therapy may be quantified on a semi-quantitative scale, for example 0 to 5, where 0 represents absence, 1 to 4 represent identifiable increases in severity, and 5 represents maximum severity. Clinical trials often use a 1 to 5 scale where: 1 represents a mild adverse event (side effect); 2 represents a moderate adverse event (side effect); 3 represents a severe adverse event (side effect); 4 represents a life-threatening or disabling adverse event (side effect); and 5 represents death related to adverse event (side effect). Alternatively, a side effect or symptom of IL-2 therapy may be quantified as a binary event, i.e., presence or absence, 0 or 1. Other semi-quantitative scales will be readily apparent to the person skilled in the art. In another embodiment, a side effect or symptom of IL-2 therapy may be quantified on a quantitative scale, for instance: mass per volume such as mass of cytokine per volume of tissue fluid; temperature; duration; rate; enzyme activity; oxygen saturation; and so on. The person skilled in the art will readily understand how to assess ad quantify any side effect or symptom of IL-2 therapy and be able to do so without difficulty or undue burden. For example, the person skilled in the art will be able to measure: a cytokine concentration in plasma or serum; temperature (fever); heart rate (tachycardia); blood pressure (hypotension); cardiac dysfunction; renal impairment; serum or plasma enzyme concentrations (hepatic function); and so on. Any quantification of a side effect or symptom of IL-2 therapy may be compared to a control, for example a healthy control subject not receiving IL-2 therapy, or comparing to other control subjects receiving, for example, standard high dose rhIL-2 therapy.

A “lowered risk” of a side effect of IL-2 therapy may be about a 1% decrease, about a 2% decrease, about a 3% decrease, about a 4% decrease, about a 5% decrease, about a 6% decrease, about a 7% decrease, about an 8% decrease, about a 9% decrease, about a 10% decrease, about a 20% decrease, about a 30% decrease, about a 40% decrease, about a 50% decrease, about a 60% decrease, about a 70% decrease, about an 80% decrease, about a 90% decrease, about a 100%, decrease in the manifestation of side effects or symptom of IL-2 therapy as compared to, for example high dose rhIL-2 therapy. Alternatively, treating a side effect or symptom of IL-2 therapy may be about a 2-fold, about a 3-fold, about a 4-fold, about a 5-fold, about a 6-fold, about a 7-fold, about an 8-fold, about, a 9-fold, about a 10-fold, or more decrease in the side effect or symptom of IL-2 therapy. It follows that “less severe side effects” refers to such a decrease in the side effect or symptom of IL-2 therapy.

Preferably the dosing regimen of the Fusion Protein in accordance with the invention reduces the frequency and severity of capillary leak syndrome (CLS) also referred to herein as vascular leak syndrome (VLS).

The risks of other side effects often associated with rhIL-2 immunotherapy that may be lowered by the treatment regimens of the invention include but are not limited to cytokine-release syndrome (CRS). CRS is a serious side effect of immunotherapy having symptoms that may overlap clinically with those of CLS and yet may cause symptoms that are entirely different from CRS. CRS is thought to result from proliferating T cells that release large quantities of cytokines, including IL-6, IFN-γ, TNF, IL-2, IL-2-receptor a, IL-8, IL-10, and GMCSF. Patients with CRS may experience any one or more of fever, cardiovascular symptoms including tachycardia, hypotension, arrhythmias, decreased cardiac ejection fraction, pulmonary symptoms including edema, hypoxia, dyspnea, and pneumonitis, acute renal injury usually caused by reduced renal perfusion, hepatic and gastrointestinal symptoms including elevated serum transaminases and bilirubin, diarrhea, colitis, nausea, and abdominal pain, hematologic symptoms including cytopenia such as grade 3-4 anemia, thrombocytopenia, leukopenia, neutropenia, and lymphopenia, derangements of coagulation including prolongation of the prothrombin time and activated partial thromboplastin time (PTT), D-dimer elevation, low fibrinogen, disseminated intravascular coagulation, macrophage activation syndrome (MAS), hemorrhage, B-cell aplasia, and hypogammaglobulinemia, infectious diseases including bacteremia, Salmonella, urinary tract infections, viral infections such as influenza, respiratory syncytial virus, and herpes zoster virus, musculoskeletal symptoms including elevated creatine kinase, myalgias and weakness, neurological symptoms including delirium, confusion, and seizure.

Administration of SEQ ID NO: 1 has been shown to induce lower levels of inflammatory cytokines as compared to for example, rhIL-2 in mice.

Administration of SEQ ID NO: 1 has also been shown to induce higher levels of desirable cytokines such as IFN while inducing lower levels of inflammatory cytokines such as IL-6 in humans.

MAS overlaps clinically with CRS with subjects potentially experiencing hepatosplenomegaly, lymphadenopathy, pancytopenia, liver dysfunction, disseminated intravascular coagulation, hypofibrinogenemia, hyperferritinemia, and hypertriglyceridemia. Like CRS, subjects with MAS exhibit elevated levels of cytokines, including IFN-γ and GMCSF.

Another side effect of immunotherapy including IL-2 therapy is tumor lysis syndrome (TLS), which occurs when the contents of cells are released as a result of therapy causing cell death, most often with lymphoma and leukemia. TLS is characterized by blood ion and metabolite imbalance, and symptoms include nausea, vomiting, acute uric acid nephropathy, acute kidney failure, seizures, cardiac arrhythmias, and death.

Neurotoxicity may result from immunotherapy including IL-2 therapy and symptoms may include cerebral edema, delirium, hallucinations, dysphasia, akinetic mutism, headache, confusion, alterations in wakefulness, ataxia, apraxia, facial nerve palsy, tremor, dysmetria, and seizure.

Patients undergoing IL-2 immunotherapy may experience one or more side effects or symptoms that are not necessarily caused by CLS, CRS, MAS or TLS including anemia, aphasia, arrhythmia, arthralgia, back pain, blood and bone marrow disorders, blood and lymphatic system disorders, cardiac disorders, chills, coagulation disorders, colitis, confused state, constitutional symptoms, cough, decreased appetite, diarrhea, disorientation, dizziness, dyspnea, encephalopathy, fatigue, fever, gastrointestinal disorders, general cardiovascular disorders, hemorrhage, hepatic disorders, hyperglycemia, hypokalemia, hypothyroidism, increased ALT, increased AST, increased C-reactive protein, infection febrile neutropenia, leukopenia, malaise, abnormal metabolic laboratory-testing results, metabolism nutrition disorders, mucosal inflammation, musculoskeletal disorders, myalgia nausea, nervous system disorders, neurologic disorders, neutropenia edema, pain, palmar-plantar erythrodysesthesia, paresthesia, pneumonia, pruritus, pulmonary disorders, pyrexia, rash, renal genitourinary disorders, respiratory disorders, skin and subcutaneous tissue disorders, somnolence, speech disorders, sweats thoracic mediastinal disorders, thrombocytopenia, tremor, tumor flare, tumor lysis syndrome, vascular disorders, and vomiting.

### Cancer Indications

The treatment regimens of the invention using the fusion protein of SEQ ID NO: 1 are useful in the treatment of many types of cancer. The term “cancer” as used herein, shall be given its ordinary meaning, as a general term for diseases in which abnormal cells divide without control. In particular, and in the context of the embodiments of the present invention, cancer refers to angiogenesis-related cancer. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Glioblastoma, Childhood; Glioblastoma, Adult; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult. Benign tumors have less of a tendency to invade and are less likely to metastasize.

The term “reducing a tumor” as used herein refers to a reduction in the size or volume of a tumor mass, a decrease in the number of metastasized tumors in a subject, a decrease in the proliferative status (the degree to which the cancer cells are multiplying) of the cancer cells, and the like.

The treatment regimens of the invention are particularly suited for treating solid tumors including but not limited to: lymphomas, melanoma, renal cell carcinoma (RCC), advanced solid tumors, tumors that have previously been treated with therapeutic therapy but remain refractory to previous therapies. Preferably, the treatment regimens of the invention are particularly suited for treating solid tumors including but not limited to: lymphomas, melanoma, renal cell carcinoma (RCC), hepatic cell carcinoma (HCC), non-small cell lung cancer (NSCLC), small cell lung cancer (SCLC), squamous cell carcinoma of the head and neck (SCCHN) and including advanced solid tumors and tumors that have previously been treated with anti-cancer therapy but remain refractory to previous therapies.

### Complementary Immunotherapies and Other Combination Therapies

While the fusion protein of SEQ ID NO: 1 may be used as a monotherapy in the treatment regimens in accordance with the invention, the combination of the fusion protein of SEQ ID NO: 1 with other anticancer treatments in the context of the invention is also contemplated. Other therapeutic treatment regimens include other therapeutic immunotherapies such as adoptive cell transfer regimens, antigen-specific vaccination, inhibition of DNA repair proteins (e.g. inhibitors of the nucleic enzyme poly(adenosine 5′-diphospho-ribose) polymerase [“poly(ADP-ribose) polymerase” PARP inhibitors”) and blockade of immune checkpoint inhibitory molecules, for example cytotoxic T lymphocyte-associated antigen 4 (CTLA-4) and programmed death 1 (PD-1) antibodies.

Immune checkpoint proteins regulate T cell function in the immune system. T cells play a central role in cell-mediated immunity. Immune checkpoint proteins interact with specific ligands that send a signal into the T cell and essentially switch off or inhibit T cell function. Cancer cells take advantage of this system by driving high levels of expression of immune checkpoint proteins on their surface that results in control of the T cells expressing immune checkpoint proteins on the surface of T cells that enter the tumor microenvironment, thus suppressing the anticancer immune response. As such, inhibition of immune checkpoint proteins by agents referred to herein as “immune checkpoint protein (ICP) inhibitors” would result in restoration of T cell function and an immune response to the cancer cells. Examples of immune checkpoint proteins include, but are not limited to: CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, OX40, B-7 family ligands or a combination thereof. Preferably, the immune checkpoint inhibitor interacts with a ligand of an immune checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, OX40, A2aR, B-7 family ligands or a combination thereof. Preferably, the immune checkpoint inhibitor is a biologic therapeutic or a small molecule. Preferably, the immune checkpoint inhibitor is a monoclonal antibody, a humanized antibody, a fully human antibody, a fusion protein or a combination thereof. Preferably, the PD1 immune checkpoint inhibitor comprises one or more anti-PD-1 antibodies, including nivolumab and pembrolizumab.

The combination therapy methods described herein include administering at least one immune checkpoint inhibitor in combination with the fusion protein of SEQ ID NO: 1. The invention is not limited to any specific immune checkpoint inhibitor so long as the immune checkpoint inhibitor inhibits one or more activities of the target immune checkpoint proteins when administered in an effective amount as monotherapy or in combination with the fusion protein of SEQ ID NO: 1. In some instances, due to, for example, synergistic effects, minimal inhibition of the immune checkpoint protein by the immune checkpoint inhibitor may be sufficient in the presence of SEQ ID NO: 1. Many immune checkpoint inhibitors are known in the art.

Exemplary PD-1/PD-L1 based immune checkpoint inhibitors include antibody-based therapeutics. Exemplary treatment methods that employ PD-1/PD-L1 based immune checkpoint inhibition are described in U.S. Pat. Nos. 8,728,474 and 9,073,994, and EP U.S. Pat. No. 1,537,878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (OPDIVO®, Bristol-Myers Squibb Co.), pembrolizumab (KEYTRUDA®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552, 154, and 8,217,149. Exemplary anti-PD-L1 antibodies include, for example, atezolizumab (TECENTRIQ®, Genentech), durvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

Preferably, a method or composition of the invention is administered in combination with (i) a PD-1 or PD-L1 inhibitor, e.g., a PD-1 or PD-L1 inhibitor disclosed herein, and (ii) CTLA-4 inhibitor, e.g., a CTLA-4 inhibitor disclosed herein.

Examples of FDA approved immune checkpoint protein inhibitors includes:
ipilimumab (YERVOY®)
pembrolizumab (KEYTRUDA®)
atezolizumab (TECENTRIQ®)
durvalumab (IMFINZ®)
avelumab (BAVENCIO®)
nivolumab (OPDIVO®).

A preferred treatment regimen of the invention combines the fusion protein of SEQ ID NO: 1 administered in accordance with the invention with the immune checkpoint inhibitor, pembrolizumab. Preferably, pembrolizumab is administered on the first day of each treatment cycle of the treatment regimen according to the invention. Preferably 200 mg of pembrolizumab is administered in accordance with manufacturer's recommendations, generally once every three weeks or 21 days.

Treatment regimens with the fusion protein of SEQ ID NO: 1 in accordance with the invention may also be combined with other therapeutic agents and/or anti-cancer agents in addition to, or instead of, immune checkpoint inhibitors. Preferably, the therapeutic agent and/or anti-cancer agent is an antibody. Preferably, the therapeutic agent is a therapeutic protein. Preferably, the therapeutic agent is a small molecule. Preferably the anticancer agent is an antigen. Preferably, the therapeutic agent is a population of cells. Preferably, the therapeutic agent is a therapeutic antibody. Preferably the therapeutic agent is another cytotoxic and/or chemotherapeutic agent. The term “cytotoxic agent” as used herein refers to a substance that inhibits or prevents a cellular function and/or causes cell death or destruction. Chemotherapeutic agent” includes chemical compounds useful in the treatment of cancer.

### Antibodies

Preferably the administration of SEQ ID NO: 1 may be combined with a therapeutic antibody. Methods of producing antibodies, and antigen-binding fragments thereof, are well known in the art and are disclosed in, e.g., U.S. Pat. No. 7,247,301, US2008/0138336, and U.S. Pat. No. 7,923,221, all of which are herein incorporated by reference in their entirety. Therapeutic antibodies that can be used in the methods of the present invention include, but are not limited to, any of the art-recognized therapeutic antibodies that are approved for use, in clinical trials, or in development for clinical use. In some embodiments, more than one therapeutic antibody can be included in the combination therapy of the present invention.

Non-limiting examples of therapeutic antibodies include the following, without limitation:
trastuzumab (HERCEPTIN™. by Genentech, South San Francisco, Calif.), which is used to treat HER-2/neu positive breast cancer or metastatic breast cancer;
bevacizumab (AVASTIN™ by Genentech), which is used to treat colorectal cancer, metastatic colorectal cancer, breast cancer, metastatic breast cancer, non-small cell lung cancer, or renal cell carcinoma;
rituximab (RITUXAN™ by Genentech), which is used to treat non-Hodgkin's lymphoma or chronic lymphocytic leukemia;
pertuzumab (OMNITARG™ by Genentech), which is used to treat breast cancer, prostate cancer, non-small cell lung cancer, or ovarian cancer;
cetuximab (ERBITUX™ by ImClone Systems Incorporated, New York, N.Y.), which can be used to treat colorectal cancer, metastatic colorectal cancer, lung cancer, head and neck cancer, colon cancer, breast cancer, prostate cancer, gastric cancer, ovarian cancer, brain cancer, pancreatic cancer, esophageal cancer, renal cell cancer, prostate cancer, cervical cancer, or bladder cancer;
IMC-1C11 (ImClone Systems Incorporated), which is used to treat colorectal cancer, head and neck cancer, as well as other potential cancer targets;
tositumomab and tositumomab and iodine I¹³¹ (BEXXAR™ by Corixa Corporation, Seattle, Wash.), which is used to treat non-Hodgkin's lymphoma, which can be CD20 positive, follicular, non-Hodgkin's lymphoma, with and without transformation, whose disease is refractory to Rituximab and has relapsed following chemotherapy;
In¹¹¹ ibirtumomab tiuxetan; Y⁹⁰ ibirtumomab tiuxetan; I¹¹¹ ibirtumomab tiuxetan and Y⁹⁰ ibirtumomab tiuxetan (ZEVALIN™ by Biogen Idec, Cambridge, Mass.), which is used to treat lymphoma or non-Hodgkin's lymphoma, which can include relapsed follicular lymphoma; relapsed or refractory, low grade or follicular non-Hodgkin's lymphoma; or transformed B-cell non-Hodgkin's lymphoma;
EMD 7200 (EMD Pharmaceuticals, Durham, N.C.), which is used for treating for treating non-small cell lung cancer or cervical cancer;
SGN-30 (a genetically engineered monoclonal antibody targeted to CD30 antigen by Seattle Genetics, Bothell, Wash.), which is used for treating Hodgkin's lymphoma or non-Hodgkin's lymphoma;
SGN-15 (a genetically engineered monoclonal antibody targeted to a Lewisγ-related antigen that is conjugated to doxorubicin by Seattle Genetics), which is used for treating non-small cell lung cancer;
SGN-33 (a humanized antibody targeted to CD33 antigen by Seattle Genetics), which is used for treating acute myeloid leukemia (AML) and myelodysplastic syndromes (MDS);
SGN-40 (a humanized monoclonal antibody targeted to CD40 antigen by Seattle Genetics), which is used for treating multiple myeloma or non-Hodgkin's lymphoma;
SGN-35 (a genetically engineered monoclonal antibody targeted to a CD30 antigen that is conjugated to auristatin E by Seattle Genetics), which is used for treating non-Hodgkin's lymphoma;
SGN-70 (a humanized antibody targeted to CD70 antigen by Seattle Genetics), that is used for treating renal cancer and nasopharyngeal carcinoma;
SGN-75 (a conjugate comprised of the SGN70 antibody and an Auristatin derivative by Seattle Genetics); and
SGN-17/19 (a fusion protein containing antibody and enzyme conjugated to melphalan prodrug by Seattle Genetics), which is used for treating melanoma or metastatic melanoma.

The therapeutic antibodies to be used in the methods of the present invention are not limited to those described herein. For example, the following approved therapeutic antibodies can also be used in the methods of the invention: brentuximab vedotin (ADCETRIS™) for anaplastic large cell lymphoma and Hodgkin lymphoma, ipilimumab (MDX-101; YERVOY™) for melanoma, ofatumumab (ARZERRA™) for chronic lymphocytic leukemia, panitumumab (VECTIBIX™) for colorectal cancer, alemtuzumab (CAMPATH™) for chronic lymphocytic leukemia, ofatumumab (ARZERRA™) for chronic lymphocytic leukemia, gemtuzumab ozogamicin (MYLOTARG™) for acute myelogenous leukemia.

Antibodies for use in accordance with the invention can also target molecules expressed by immune cells, such as, but not limited to, tremelimumab (CP-675,206) and ipilimumab (MDX-010) which targets CTLA4 and has the effect of tumor rejection, protection from re-challenge, and enhanced tumor-specific T cell responses; OX86 which targets OX40 and increases antigen-specific CD8+ T cells at tumor sites and enhances tumor rejection; CT-011 which targets PD 1 and has the effect of maintaining and expanding tumor specific memory T cells and activates NK cells; BMS-663513 which targets CD137 and causes regression of established tumors, as well as the expansion and maintenance of CD8+ T cells, and daclizumab (ZENAPAX™) which targets CD25 and causes transient depletion of CD4+CD25+FOXP3+Tregs and enhances tumor regression and increases the number of effector T cells. A more detailed discussion of these antibodies can be found in, e.g., Weiner et al., Nature Rev. Immunol 2010; 10:317-27.

Preferably, the antibody is a pro-inflammatory and/or pro-tumorigenic cytokine targeting antibody including, but not limited to, anti-TNF antibodies, anti-IL-1Ra receptor targeting antibodies, anti-IL-1 antibodies, anti-IL-6 receptor antibodies, and anti-IL-6 antibodies. Preferably antibodies include those that target pro-inflammatory T helper type 17 cells (TH17).

The therapeutic antibody can be an antigen-binding fragment of an antibody; a complex comprising an antibody; or a conjugate comprising an antibody. The antibody can optionally be chimeric or humanized or fully human.

### Therapeutic Proteins and Polypeptides

Preferably the methods of the invention include administration of the fusion protein of SEQ ID NO: 1 in accordance with the treatment regimen of the invention in combination with a therapeutic protein or peptide. Therapeutic proteins that are effective in treating cancer are well known in the art. Preferably, the therapeutic polypeptide or protein is a “suicide protein” that causes cell death by itself or in the presence of other compounds.

A representative example of such a suicide protein is thymidine kinase of the herpes simplex virus. Additional examples include thymidine kinase of varicella zoster virus, the bacterial gene cytosine deaminase (which converts 5-fluorocytosine to the highly toxic compound 5-fluorouracil), p450 oxidoreductase, carboxypeptidase G2, beta-glucuronidase, penicillin-V-amidase, penicillin-G-amidase, beta-lactamase, nitroreductase, carboxypeptidase A, linamarase (also referred to as β-glucosidase), the E. coli gpt gene, and the E. coli Deo gene, although others are known in the art. In some embodiments, the suicide protein converts a prodrug into a toxic compound.

As used herein, “prodrug” means any compound useful in the methods of the present invention that can be converted to a toxic product, i.e. toxic to tumor cells. The prodrug is converted to a toxic product by the suicide protein. Representative examples of such prodrugs include: ganciclovir, acyclovir, and FIAU (1-(2-deoxy-2-fluoro-β-D-arabinofuranosyl)-5-iod-ouracil) for thymidine kinase; ifosfamide for oxidoreductase; 6-methoxypurine arabinoside for VZV-TK; 5-fluorocytosine for cytosine deaminase; doxorubicin for beta-glucuronidase; CB 1954 and nitrofurazone for nitroreductase; and N-(Cyanoacetyl)-L-phenylalanine or N-(3-chloropropionyl)-L-phenylalanine for carboxypeptidase A. The prodrug may be administered readily by a person having ordinary skill in this art. A person with ordinary skill would readily be able to determine the most appropriate dose and route for the administration of the prodrug.

Preferably the therapeutic protein or polypeptide, is a cancer suppressor, for example p53 or Rb, or a nude acid encoding such a protein or polypeptide. Those of skill know of a wide variety of such cancer suppressors and how to obtain them and/or the nucleic acids encoding them.

Other examples of anti-cancer/therapeutic proteins or polypeptides include pro-apoptotic therapeutic proteins and polypeptides, for example, p15, p16, or p21^{WAF-1}.

Cytokines, and nucleic acid encoding them may also be used as therapeutic proteins and polypeptides. Examples include: GM-CSF (granulocyte macrophage colony stimulating factor); TNF-alpha (Tumor necrosis factor alpha); Interferons including, but not limited to, IFN-alpha and IFN-gamma; and Interleukins including, but not limited to, Interleukin-1 (IL-1), Interleukin-Beta (IL-beta), Interleukin-2 (IL-2), Interleukin-4 (IL-4), Interleukin-5 (IL-5), Interleukin-6 (IL-6), Interleukin-7 (IL-7), Interleukin-8 (IL-8), Interleukin-10 (IL-10), Interleukin-12 (IL-12), Interleukin-13 (IL-13), Interleukin-14 (IL-14), Interleukin-15 (IL-15), Interleukin-16 (IL-16), Interleukin-18 (IL-18), Interleukin-23 (IL-23), Interleukin-24 (IL-24), although other embodiments are known in the art.

Additional examples of cytocidal genes includes, but is not limited to, mutated cyclin G1 genes. By way of example, the cytocidal gene may be a dominant negative mutation of the cyclin G1 protein (e.g., WO/01/64870).

### Vaccines

Preferably, the therapeutic regimens of the invention include administration of a fusion protein of SEQ ID NO: 1 in combination with administration of a cancer vaccine for stimulating a cancer specific-immune response, e.g., innate and adaptive immune responses, for generating host immunity against a cancer (see, e.g., Overwijk, et al. Journal of Experimental Medicine 2008; 198:569-80). Illustrative vaccines include, but are not limited to, for example, antigen vaccines, whole cell vaccines, dendritic cell vaccines, and DNA vaccines. Depending upon the particular type of vaccine, the vaccine composition may include one or more suitable adjuvants known to enhance a subject's immune response to the vaccine.

The vaccine may, for example, be cellular based, i.e., created using cells from the patient's own cancer cells to identify and obtain an antigen. Exemplary vaccines include tumor cell-based and dendritic-cell based vaccines, where activated immune cells from the subject are delivered back to the same subject, along with other proteins, to further facilitate immune activation of these tumor antigen primed immune cells. Tumor cell-based vaccines include whole tumor cells and gene-modified tumor cells. Whole tumor cell vaccines may optionally be processed to enhance antigen presentation, e.g., by irradiation of either the tumor cells or tumor lysates). Vaccine administration may also be accompanied by adjuvants such as bacillus calmette-guerin (BCG) or keyhole limpet hemocyanin (KLH), depending upon the type of vaccine employed. Plasmid DNA vaccines may also be used and can be administered via direct injection or biolistically. Also contemplated for use are peptide vaccines, viral gene transfer vector vaccines, and antigen-modified dentritic cells (DCs).

Preferably the vaccine is a therapeutic cancer peptide-based vaccine. Peptide vaccines can be created using known sequences or from isolated antigens from a subject's own tumor(s) and include neoantigens and modified antigens. Illustrative antigen-based vaccines include those where the antigen is a tumor-specific antigen. For example, the tumor-specific antigen may be selected from a cancer-testis antigen, a differentiation antigen, and a widely occurring over-expressed tumor associated antigen, among others. Recombinant peptide vaccines, based on peptides from tumor-associated antigens, when used in the instant method, may be administered or formulated with, an adjuvant or immune modulator. Illustrative antigens for use in a peptide-based vaccine include, but are not limited to, the following, since this list is meant to be purely illustrative. For example, a peptide vaccine may comprise a cancer-testis antigen such as MAGE, BAGE, NY-ESO-1 and SSX-2, encoded by genes that are normally silenced in adult tissues but transcriptionally reactivated in tumor cells. Alternatively, the peptide vaccine may comprise a tissue differentiation associated antigen, i.e., an antigen of normal tissue origin and shared by both normal and tumorous tissue. For example, the vaccine may comprise a melanoma-associated antigen such as gp100, Melan-A/Mart-1, MAGE-3, or tyrosinase; or may comprise a prostate cancer antigen such as PSA or PAP. The vaccine may comprise a breast cancer-associated antigen such as mammaglobin-A. Other tumor antigens that may be comprised in a vaccine for use in the instant method include, for example, CEA, MUC-1, HER1/Nue, hTERT, ras, and B-raf. Other suitable antigens that may be used in a vaccine include SOX-2 and OCT-4, associated with cancer stem cells or the EMT process.

Antigen vaccines include multi-antigen and single antigen vaccines. Exemplary cancer antigens may include peptides having from about 5 to about 30 amino acids, or from about 6 to 25 amino acids, or from about 8 to 20 amino acids.

As described above, an immunostimulatory adjuvant (different from RSLAIL-2) may be used in a vaccine, in particular, a tumor-associated antigen-based vaccine, to assist in generating an effective immune response. For example, a vaccine may incorporate a pathogen-associated molecular pattern (PAMP) to assist in improving immunity. Additional suitable adjuvants include monophosphoryl lipid A, or other lipopolysaccharides; toll-like receptor (TLR) agonists such as, for example, imiquimod, resiquimod (R-848), TLR3, IMO-8400, and rintatolimod. Additional adjuvants suitable for use include heat shock proteins.

A genetic vaccine typically uses viral or plasmid DNA vectors carrying expression cassettes. Upon administration, they transfect somatic cells or dendritic cells as part of the inflammatory response to thereby result in cross-priming or direct antigen presentation. Preferably, a genetic vaccine is one that provides delivery of multiple antigens in one immunization. Genetic vaccines include DNA vaccines, RNA vaccines and viral-based vaccines.

DNA vaccines for use in the instant methods are bacterial plasmids that are constructed to deliver and express tumor antigen. DNA vaccines may be administered by any suitable mode of administration, e.g., subcantaneous or intradermal injection, but may also be injected directly into the lymph nodes. Additional modes of delivery include, for example, gene gun, electroporation, ultrasound, laser, liposomes, microparticles and nanoparticles.

Preferably, the vaccine comprises a neoantigen, or multiple neoantigens. Preferably, the vaccine is a neoantigen-based vaccine. Preferably a neoantigen-based vaccine (NBV) composition may encode multiple cancer neoantigens in tandem, where each neoantigen is a polypeptide fragment derived from a protein mutated in cancer cells. For instance, a neoantigenic vaccine may comprise a first vector comprising a nucleic acid construct encoding multiple immunogenic polypeptide fragments, each of a protein mutated in cancer cells, where each immunogenic polypeptide fragment comprises one or more mutated amino acids flanked by a variable number of wild type amino acids from the original protein, and each polypeptide fragment is joined head-to-tail to form an immunogenic polypeptide. The lengths of each of the immunogenic polypeptide fragments forming the immunogenic polypeptide can vary.

Viral gene transfer vector vaccines may also be used; in such vaccines, recombinant engineered virus, yeast, bacteria or the like is used to introduce cancer-specific proteins to the patient's immune cells. In a vector-based approach, which can be tumor lytic or non-tumor lytic, the vector can increase the efficiency of the vaccine due to, for example, its inherent immunostimulatory properties. Illustrative viral-based vectors include those from the poxviridae family, such as vaccinia, modified vaccinia strain Ankara and avipoxviruses. Also suitable for use is the cancer vaccine, PROSTVAC, containing a replication-competent vaccinia priming vector and a replication-incompetent fowlbox-boosting vector. Each vector contains transgenes for PSA and three co-stimulatory molecules, CD80, CD54 and CD58, collectively referred to as TRICOM. Other suitable vector-based cancer vaccines include Trovax and TG4010 (encoding MUC1 antigen and IL-2). Additional vaccines for use include bacteria and yeast-based vaccines such as recombinant Listeria monocytogenes and Saccharomyces cerevisae.

The foregoing vaccines may be combined and/or formulated with adjuvants and other immune boosters to increase efficacy. Depending upon the particular vaccine, administration may be either intratumoral or non-intratumoral (i.e., systemic).

Other cancer antigens that can be used in vaccinations include, but are not limited to, (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

The cancer antigen may be an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen.

In another embodiment, the cancer antigen is a lymphoma antigen (e.g., non-Hodgkin's lymphoma or Hodgkin's lymphoma), a B-cell lymphoma cancer antigen, a leukemia antigen, a myeloma (i.e., multiple myeloma or plasma cell myeloma) antigen, an acute lymphoblastic leukemia antigen, a chronic myeloid leukemia antigen, or an acute myelogenous leukemia antigen. The described cancer antigens are only exemplary, and that any cancer antigen can be targeted in the present invention.

Preferably, the cancer antigen is a mucin-1 protein or peptide (MUC-1) that is found on all human adenocarcinomas: pancreas, colon, breast, ovarian, lung, prostate, head and neck, including multiple myelomas and some B cell lymphomas. Patients with inflammatory bowel disease, either Crohn's disease or ulcerative colitis, are at an increased risk for developing colorectal carcinoma. MUC-1 is a type I transmembrane glycoprotein. The major extracellular portion of MUC-1 has a large number of tandem repeats consisting of 20 amino acids which comprise immunogenic epitopes. In some cancers it is exposed in an unglycosylated form that is recognized by the immune system (Gendler et al., J Biol Chem 1990; 265:15286-15293).

In another embodiment, the cancer antigen is a mutated B-Raf antigen, which is associated with melanoma and colon cancer. The vast majority of these mutations represent a single nucleotide change of T-A at nucleotide 1796 resulting in a valine to glutamic acid change at residue 599 within the activation segment of B-Raf. Raf proteins are also indirectly associated with cancer as effectors of activated Ras proteins, oncogenic forms of which are present in approximately one-third of all human cancers. Normal non-mutated B-Raf is involved in cell signaling, relaying signals from the cell membrane to the nucleus. The protein is usually only active when needed to relay signals. In contrast, mutant B-Raf has been reported to be constantly active, disrupting the signaling relay (Mercer and Pritchard, Biochim Biophys Acta (2003) 1653 (1): 25-40; Sharkey et al., Cancer Res. (2004) 64 (5): 1595-1599).

Preferably, the cancer antigen is a human epidermal growth factor receptor-2 (HER-2/neu) antigen. Cancers that have cells that overexpress HER-2/neu are referred to as HER-2/neu⁺ cancers. Exemplary HER-2/neu⁺ cancers include prostate cancer, lung cancer, breast cancer, ovarian cancer, pancreatic cancer, skin cancer, liver cancer (e.g., hepatocellular adenocarcinoma), intestinal cancer, and bladder cancer.

HER-2/neu has an extracellular binding domain (ECD) of approximately 645 aa, with 40% homology to epidermal growth factor receptor (EGFR), a highly hydrophobic transmembrane anchor domain (TMD), and a carboxyterminal intracellular domain (ICD) of approximately 580 aa with 80% homology to EGFR. The nucleotide sequence of HER-2/neu is available at GENBANK™. Accession Nos. AH002823 (human HER-2 gene, promoter region and exon 1); M16792 (human HER-2 gene, exon 4): M16791 (human HER-2 gene, exon 3); M16790 (human HER-2 gene, exon 2); and M16789 (human HER-2 gene, promoter region and exon 1). The amino acid sequence for the HER-2/neu protein is available at GENBANK™. Accession No. AAA58637. Based on these sequences, one skilled in the art could develop HER-2/neu antigens using known assays to find appropriate epitopes that generate an effective immune response.

Exemplary HER-2/neu antigens include p369-377 (a HER-2/neu derived HLA-A2 peptide); dHER2 (Corixa Corporation); li-Key MHC class II epitope hybrid (Generex Biotechnology Corporation); peptide P4 (amino acids 378-398); peptide P7 (amino acids 610-623); mixture of peptides P6 (amino acids 544-560) and P7; mixture of peptides P4, P6 and P7; HER2 [9₇₅₄]; and the like.

Preferably, the cancer antigen is an epidermal growth factor receptor (EGFR) antigen. The EGFR antigen can be an EGFR variant 1 antigen, an EGFR variant 2 antigen, an EGFR variant 3 antigen and/or an EGFR variant 4 antigen. Cancers with cells that overexpress EGFR are referred to as EGFR cancers. Exemplary EGFR cancers include lung cancer, head and neck cancer, colon cancer, colorectal cancer, breast cancer, prostate cancer, gastric cancer, ovarian cancer, brain cancer and bladder cancer.

Preferably, the cancer antigen is a vascular endothelial growth factor receptor (VEGFR) antigen. VEGFR is considered to be a regulator of cancer-induced angiogenesis. Cancers with cells that overexpress VEGFR are called VEGFR cancers. Exemplary VEGFR⁺ cancers include breast cancer, lung cancer, small cell lung cancer, colon cancer, colorectal cancer, renal cancer, leukemia, and lymphocytic leukemia.

Preferably, the cancer antigen is prostate-specific antigen (PSA) and/or prostate-specific membrane antigen (PSMA) that are prevalently expressed in androgen-independent prostate cancers.

Preferably, the cancer antigen is Gp-100 Glycoprotein 100 (gp 100) is a tumor-specific antigen associated with melanoma.

Preferably, the cancer antigen is a carcinoembryonic (CEA) antigen. Cancers with cells that overexpress CEA are referred to as CEA⁺ cancers. Exemplary CEA⁺ cancers include colorectal cancer, gastric cancer and pancreatic cancer. Exemplary CEA antigens include CAP-1 (i.e., CEA aa 571-579), CAP1-6D, CAP-2 (i.e., CEA aa 555-579), CAP-3 (i.e., CEA aa 87-89), CAP-4 (CEA aa 1-11), CAP-5 (i.e., CEA aa 345-354), CAP-6 (i.e., CEA aa 19-28) and CAP-7.

Preferably, the cancer antigen is carbohydrate antigen 10.9 (CA 19.9). CA 19.9 is an oligosaccharide related to the Lewis A blood group substance and is associated with colorectal cancers.

Preferably, the cancer antigen is a melanoma cancer antigen. Melanoma cancer antigens are useful for treating melanoma. Exemplary melanoma cancer antigens include MART-1 (e.g., MART-1 26-35 peptide, MART-1 27-35 peptide); MART-1/Melan A; pMel17; pMel17/gp100; gp100 (e.g., gp 100 peptide 280-288, gp 100 peptide 154-162, gp 100 peptide 457-467); TRP-1; TRP-2; NY-ESO-1; p16; beta-catenin; mum-1; and the like.

Preferably, the cancer antigen is a mutant or wild type ras peptide. The mutant ras peptide can be a mutant K-ras peptide, a mutant N-ras peptide and/or a mutant H-ras peptide. Mutations in the ras protein typically occur at positions 12 (e.g., arginine or valine substituted for glycine), 13 (e.g., asparagine for glycine), 61 (e.g., glutamine to leucine) and/or 59. Mutant ras peptides can be useful as lung cancer antigens, gastrointestinal cancer antigens, hepatoma antigens, myeloid cancer antigens (e.g., acute leukemia, myelodysplasia), skin cancer antigens (e.g., melanoma, basal cell, squamous cell), bladder cancer antigens, colon cancer antigens, colorectal cancer antigens, and renal cell cancer antigens.

In another embodiment of the invention, the cancer antigen is a mutant and/or wildtype p53 peptide. The p53 peptide can be used as colon cancer antigens, lung cancer antigens, breast cancer antigens, hepatocellular carcinoma cancer antigens, lymphoma cancer antigens, prostate cancer antigens, thyroid cancer antigens, bladder cancer antigens, pancreatic cancer antigens and ovarian cancer antigens.

The cancer antigen can be a cell, a protein, a peptide, a fusion protein, DNA encoding a peptide or protein, RNA encoding a peptide or protein, a glycoprotein, a lipoprotein, a phosphoprotein, a carbohydrate, a lipopolysaccharide, a lipid, a chemically linked combination of two or more thereof, a fusion or two or more thereof, or a mixture of two or more thereof, or a virus encoding two or more thereof, or an oncolytic virus encoding two or more thereof. In another embodiment, the cancer antigen is a peptide comprising about 6 to about 24 amino acids; from about 8 to about 20 amino acids; from about 8 to about 12 amino acids; from about 8 to about 10 amino acids; or from about 12 to about 20 amino acids. In one embodiment, the cancer antigen is a peptide having a MHC Class I binding motif or a MHC Class II binding motif. In another embodiment, the cancer antigen comprises a peptide that corresponds to one or more cytotoxic T lymphocyte (CTL) epitopes.

### Cell Therapy

The methods of the disclosure include administration of the fusion protein of SEQ ID NO: 1 in combination with administration of a therapeutic cell therapy. Cell therapies that are useful for treating cancer are well known and are disclosed in, e.g., U.S. Pat. No. 7,402,431. In a preferred embodiment, the cell therapy is T cell transplant. In a preferred method, T cells are expanded ex vivo with IL-2 prior to transplantation into a subject. Methods for cell therapies are disclosed in, e.g., U.S. Pat. No. 7,402,431, US2006/0057121, U.S. Pat. Nos. 5,126,132, 6,255,073, 5,846,827, 6,251,385, 6,194,207, 5,443,983, 6,040,177, 5,766,920, and US2008/0279836.

### Radiation Therapy

The methods of the disclosure include administration of a fusion protein of SEQ ID NO: 1 in further combination with radiation therapy. The term “radiation therapy” may be used interchangeably with the term “radiotherapy”, is a type of cancer treatment that uses beams of intense energy to kill cancer cells. Radiation therapy most often uses X-rays, but gamma rays, electron beams, or protons also can be used. The term “radiation therapy” most often refers to external beam radiation therapy. During this type of radiation, the high-energy beams come from a machine outside of the patient's body that aims the beams at a precise point on the body. Each session is quick and painless, lasting about 15 minutes. As used herein, the term “session” or “session of treatment” refers to each radiotherapy treatment. A radiation therapy “regimen” or “schedule” usually consists of a specific number of treatments given over a set period of time, depending on the type and the stage of the cancer.

### Small Molecules

The methods of the disclosure include administration of a fusion protein of SEQ ID NO: 1 in combination with administration of an anticancer small molecule. Small molecules that are effective in treating cancer are well known in the art and include antagonists of factors that are involved in tumor growth, such as EGFR, ErbB2 (also known as Her2) ErbB3, ErbB4, or TNF. Non-limiting examples include small molecule receptor tyrosine kinase inhibitors (RTKIs) that target one or more tyrosine kinase receptors, such as VEGF receptors, FGF receptors, EGF receptors and PDGF receptors.

Many therapeutic small molecule RTKIs are known in the art, including, but are not limited to, lenvatinib (LENVIMA), lucitanib, axitinib (INLYTA), vatalanib (PTK787), erlotinib (TARCEVA™), OSI-7904, ZD6474 (ZACTIMA™), ZD6126 (ANG453), ZD1839, sunitinib (SUTENT™), semaxanib (SU5416), AMG706, AG013736, Imatinib (GLEEVEC™), MLN-518, CEP-701, PKC-412, Lapatinib (GSK572016), VELCADE™, AZD2171, sorafenib (NEXAVAR™), XL880, and CHIR-265. Small molecule protein tyrosine phosphatase inhibitors, such as those disclosed in Jiang et al., Cancer Metastasis Rev. 2008; 27:263-72 are also useful for practicing the methods of the invention. Such inhibitors can target, e.g., HSP2, PRL, PTP1B, or Cdc25 phosphatases.

Small molecules that target Bcl-2/Bcl-XL, such as those disclosed in US2008/0058322, are also useful for practicing the methods of the present invention. Further exemplary small molecules for use in the present invention are disclosed in Zhang et al. Nature Reviews: Cancer 2009; 9:28-39. In particular, chemotherapeutic agents that lead to immunogenic cell death such as anthracyclins (Kepp et al., Cancer and Metastasis Reviews 2011; 30:61-9) will be well suited for synergistic effects with extended-PK IL-2.

### Other Cytotoxic and Chemotherapeutic Agents

The methods of the disclosure include administration of the fusion protein of SEQ ID NO: 1 in combination with chemotherapeutic agents, including but not limited to, alkylating agents, antitumor antibiotics, antimetabolic agents, other anti-tumor antibiotics, and plant derived agents.

Alkylating agents are drugs which impair cell function by forming covalent bonds with amino, carboxyl, sulfhydryl and phosphate groups in biologically important molecules. The most important sites of alkylation are DNA, RNA and proteins. Alkylating agents depend on cell proliferation for activity but are not cell-cycle-phase-specific. Alkylating agents suitable for use in the present invention include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitroso-ureas (e.g. BCNU, carmustine, lomustine, streptozocin), nonclassic alkylating agents (e.g., altretamine, dacarbazine, and procarbazine), and platinum compounds (e.g., carboplastin, oxaliplatin and cisplatin).

Antitumor antibiotics like adriamycin intercalate DNA at guanine-cytosine and guanine-thymine sequences, resulting in spontaneous oxidation and formation of free oxygen radicals that cause strand breakage. Other antibiotic agents suitable for use in the present invention include, but are not limited to, anthracyclines (e.g. doxorubicin, daunorubicin, epirubicin, idarubicin and anthracenedione), mitomycin C, bleomycin, dactinomycin, and plicatomycin.

Antimetabolic agents suitable for use in the present invention include but are not limited to, floxuridine, fluorouracil, methotrexate, leucovorin, hydroxyurea, thioguanine, mercaptopurine, cytarabine, pentostatin, fludarabine phosphate, cladribine, asparaginase, and gemcitabine.

Plant derived agents include taxanes, which are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis. Other plant derived agents include, but are not limited to, vincristine, vinblastine, vindesine, vinzolidine, vinorelbine, etoposide, teniposide, and docetaxel.

### Compositions for Combination Therapy

In certain embodiments, the fusion protein of SEQ ID NO: 1 is administered together (simultaneously or sequentially) with one or more additional therapeutic agents or other therapeutic agents, such as a therapeutic antibody. Preferably, the fusion protein of SEQ ID NO: 1 is administered prior to the administration of one or more therapeutic agents, such as a therapeutic antibody. Preferably, the fusion protein of SEQ ID NO: 1 is administered concurrent with the administration of one or more therapeutic agents, such as a therapeutic antibody. Preferably, the fusion protein of SEQ ID NO: 1 is administered subsequent to the administration of one or more therapeutic agents, such as a therapeutic antibody. Preferably, the SEQ ID NO: 1 and one or more therapeutic agents, such as a therapeutic antibody, are administered simultaneously. In other embodiments, the and one or more therapeutic agents, such as a therapeutic antibody, are administered sequentially. Preferably, the fusion protein of SEQ ID NO: 1 and one or more therapeutic agents, such as a therapeutic antibody, are administered within one, two, or three days of each other.

The one or more therapeutic agents may be those that serve as adjunctive therapy for cancer, such as cytokines, chemotherapeutic agents, small molecules, antigens, or therapeutic antibodies, and are well known in the art and discussed supra. Additional non-limiting examples of additional agents include GM-CSF (expands monocyte and neutrophil population), IL-7 (important for generation and survival of memory T-cells), interferon alpha, tumor necrosis factor alpha, IL-12, and therapeutic antibodies, such as anti-PD-1, anti-PD-L, anti-CTLA4, anti-CD40, anti-OX40, and anti-CD137, PARP inhibitors, antibodies. In some embodiments, the subject receives the fusion protein of SEQ ID NO: 1 and one or more therapeutic agents during a same period of prevention, occurrence of a disorder, and/or period of treatment.

Preferably, the invention provides for separate pharmaceutical compositions comprising the fusion protein of SEQ ID NO: 1 with a pharmaceutically acceptable diluent, carrier, solubilizer, emulsifier, preservative and/or adjuvant and another pharmaceutical composition comprising one or more therapeutic agents, such as a therapeutic antibody, with a pharmaceutically acceptable diluent, carrier, solubilizer, emulsifier, preservative and/or adjuvant.

Preferably, the invention provides for pharmaceutical compositions comprising the fusion protein of SEQ ID NO: 1 and one or more therapeutic or anti-cancer agents in the same composition, together with a pharmaceutically acceptable diluent, carrier, solubilizer, emulsifier, preservative and/or adjuvant.

### Kits

Also provided are kits comprising a fusion protein of SEQ ID NO: 1 formulated for SC administration, and optionally any other chemotherapeutic or anti-cancer agent. The kits are generally in the form of a physical structure housing various components, as described below, and can be utilized, for example, in practicing the methods described above. A kit can include the fusion protein of SEQ ID NO: 1 (provided in, e.g., a sterile container), which can be in the form of a pharmaceutical composition suitable for administration to a subject. The pharmaceutical composition can be provided in a form that is ready for use or in a form requiring, for example, reconstitution or dilution prior to administration. When the compositions are in a form that needs to be reconstituted by a user, the kit can also include buffers, pharmaceutically acceptable excipients, and the like, packaged with or separately from the fusion protein of SEQ ID NO: 1. When combination therapy (e.g., the fusion protein of SEQ ID NO: 1 and an immune checkpoint inhibitor(s) is contemplated, the kit can contain the several agents separately or they can already be combined in the kit. Similarly, when additional complementary therapy is required (e.g., a fusion protein of SEQ ID NO: 1, an immune checkpoint inhibitor(s), and an additional complementary therapy or agent) the kit can contain the several agents separately or two or more of them can already be combined in the kit.

A kit of the invention can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via an internet site, are provided.

### Equivalents and Scope

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein.

Any particular embodiment of the compositions of the invention; any method of production; any method of use can be excluded from any one or more claims, for any reason, whether or not related to the existence of prior art.

### Examples

### Example 1—A Less-Frequent Dosing Regimen of the Fusion Protein of SEQ ID NO: 1 Administered Intravenously as a Monotherapy and in Combination with Pembrolizumab in Subjects with Advanced Solid Tumors

The fusion protein of SEQ ID NO: 1 is a fusion of circularly permuted IL-2 and IL-2 Receptor α (IL-2Rα) designed to selectively activate the intermediate-affinity IL-2R, comprised of IL-2Rβ and γ, for activation of cytotoxic CD8⁺ T cells and NK cells. The intermediate-affinity IL-2R is expressed predominantly on effector lymphocytes, which play an important role in driving antitumor immune responses. Wild-type IL-2 activates the high-affinity IL-2R, comprised of IL-2Rα, β, and γ_{c}, driving the expansion of immunosuppressive CD4⁺ regulatory T (T_{reg}) cells at concentrations below those at which intermediate-affinity IL-2R-bearing effector cells are activated. Selective activation of the intermediate affinity IL-2R has the potential to enhance tumor killing and was shown to possess enhanced antitumor activity relative to IL-2 in murine models.

Prior clinical trials have relied on administering the fusion protein of SEQ ID NO: 1 on 5 consecutive days at the start of therapy. This administration schedule is cumbersome for patients and present logistic difficulties. A less-frequent administration schedule would be preferred (e.g., a single dose per a treatment cycle, two doses per a treatment cycle, or three doses per a treatment cycle). To that end, an optimized dosing regimen was developed with quantitative system pharmacology (QSP) modeling to predict doses that result in similar expansion of CD8+ T and NK cells achieved with the prior 3 μg/kg/day and 6 μg/kg/day qdx5 (administration on 5 consecutive days), with less frequent dosing.

### Methods

A QSP model for the fusion protein of SEQ ID NO: 1 was developed in MATLAB/Simbiology by leveraging published literature data and calibrated with in vitro pSTAT5 data and in vivo clinical data. The model was verified by comparing simulated cell counts in peripheral blood with observed clinical data and applied to predict the change in CD8+ T and NK cells in peripheral blood and in the tumor in response to IV administration of the fusion protein of SEQ ID NO: 1 given 1-dose (on day 1), 2-dose (on days 1 and 8 or days 1 and 4) or 3-dose (on days 1, 8 and 15 or days 1, 4 and 8) in a 21-day cycle.

### Results

The model-predicted time course of circulating CD8+ T and NK cells described observed clinical data from ARTISTRY-1 study reasonably well. Application of the QSP model predicted that a) a single dose of 30-40 μg/kg, b) 2 doses of 20-25 μg/kg/dose given on days 1 and 8, c) 2 doses of 15 μg/kg/dose given on days 1 and 4, d) 3 doses of 20 μg/kg/dose given on days 1, 8 and 15, and e) 3 doses of 15 μg/kg/dose given on days 1, 4 and 8, could result in CD8+ T and NK cell expansion comparable to that achieved with 6 μg/kg/day qdx5, respectively. Lower dose levels are required to achieve CD8+ T and NK cell expansion comparable to that achieved with 3 μg/kg/day qdx5.

The simulations with the model were run to match area under the curve (AUC) or maximum cell count (Rmax) of CD8+ T cells and NK cells in blood and tumor with 3 μg/kg or 6 μg/kg IV qdx5 (5 days on, 16 days off) in a 21 day-cycle. The simulations assumed that 3 μg/kg and 6 μg/kg showed a “stable disease” condition. Simulations were run for the following 21-day cycles:
3 dose/21-day cycle: 1) dosed on Day 1, 4, and 8; or 2) dosed on Day 1, 8, and 15.
2 dose/21-day cycle: 1) dosed on Day 1 and 4; or 2) dosed on Day 1 and 8.
1 dose/21-day cycle: dosed on Day 1.

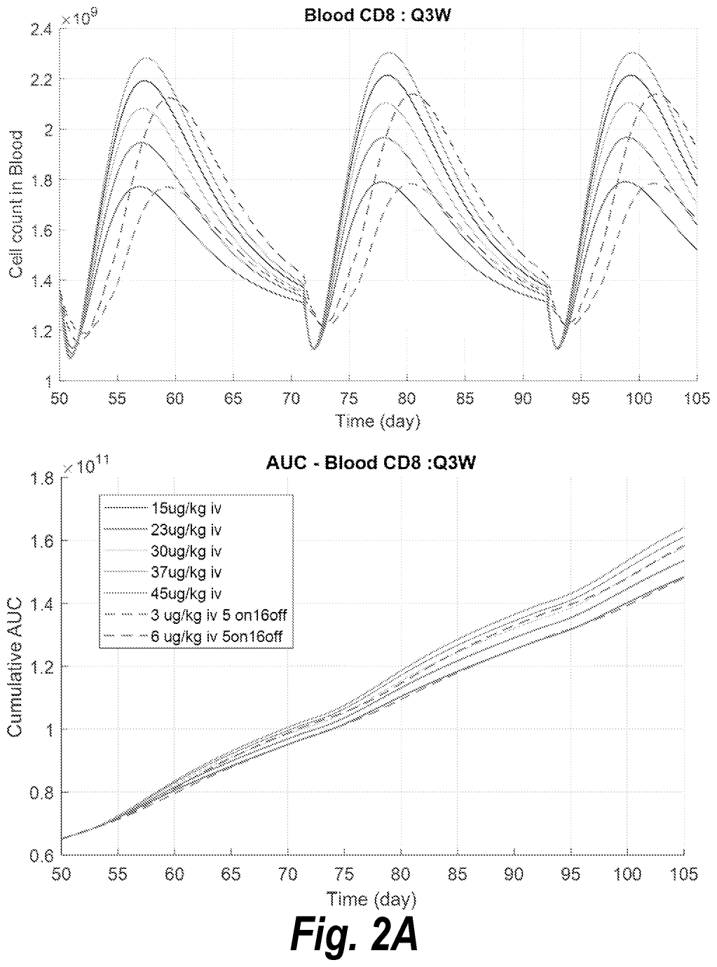

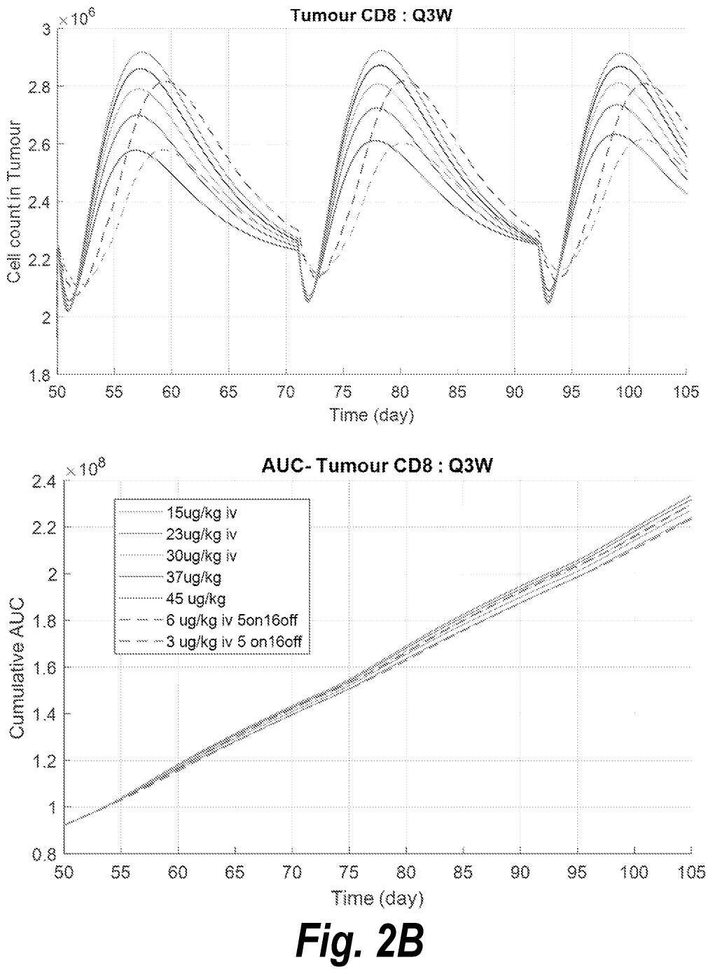

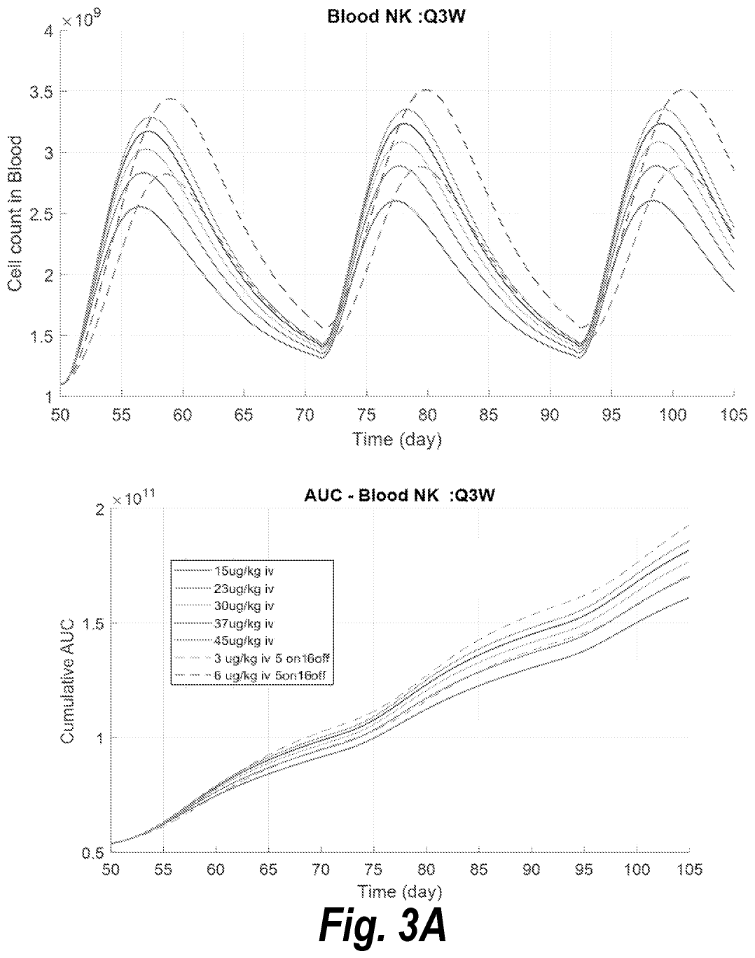

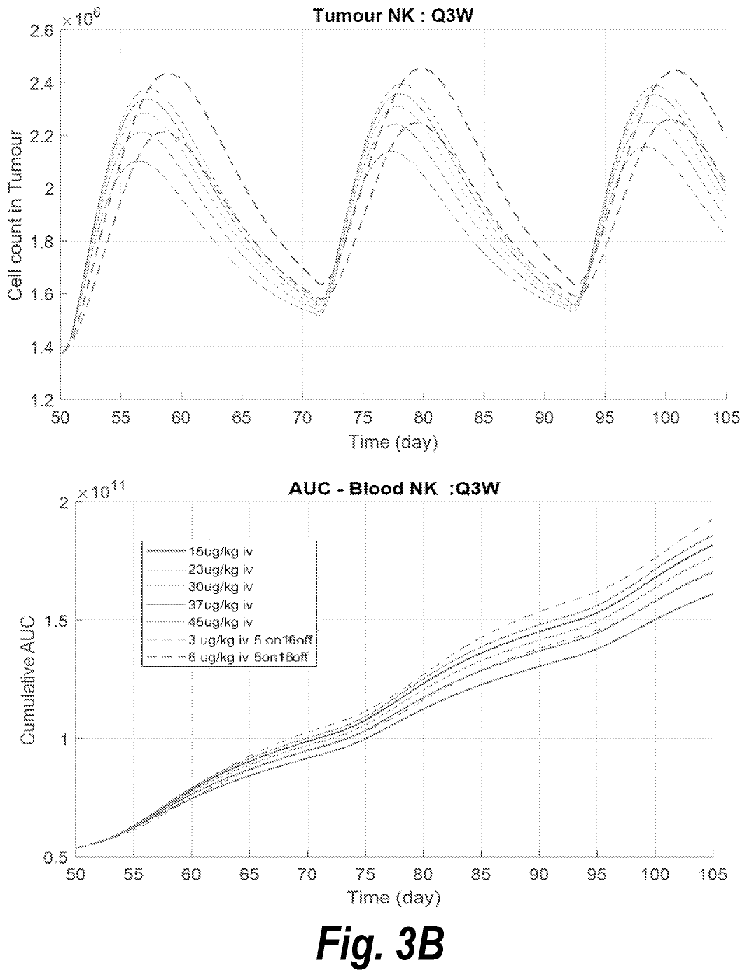

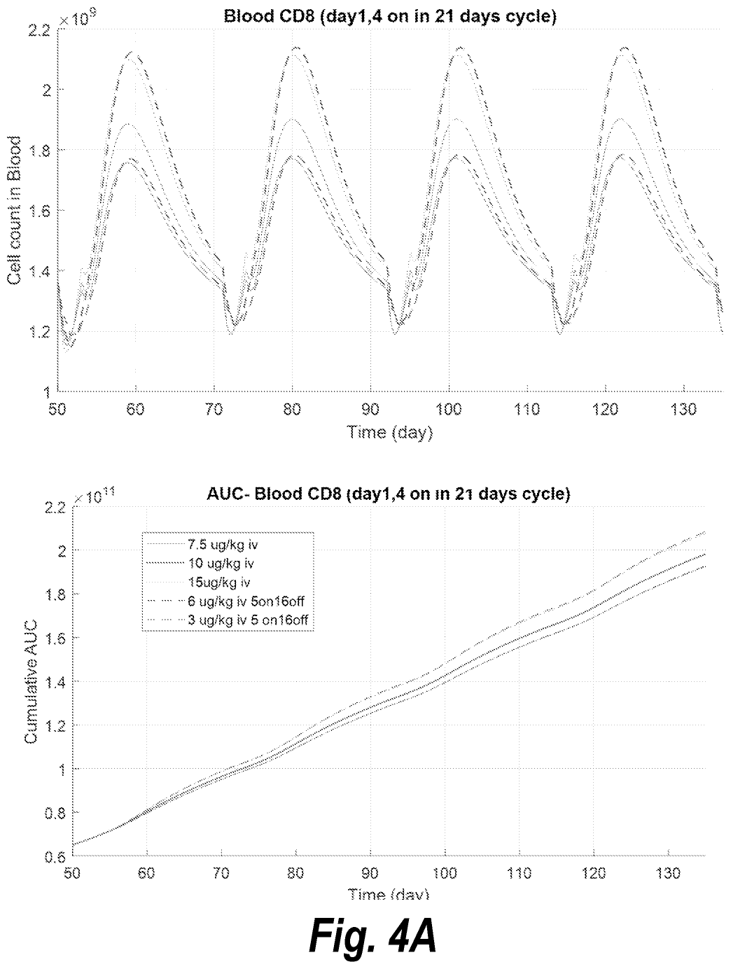

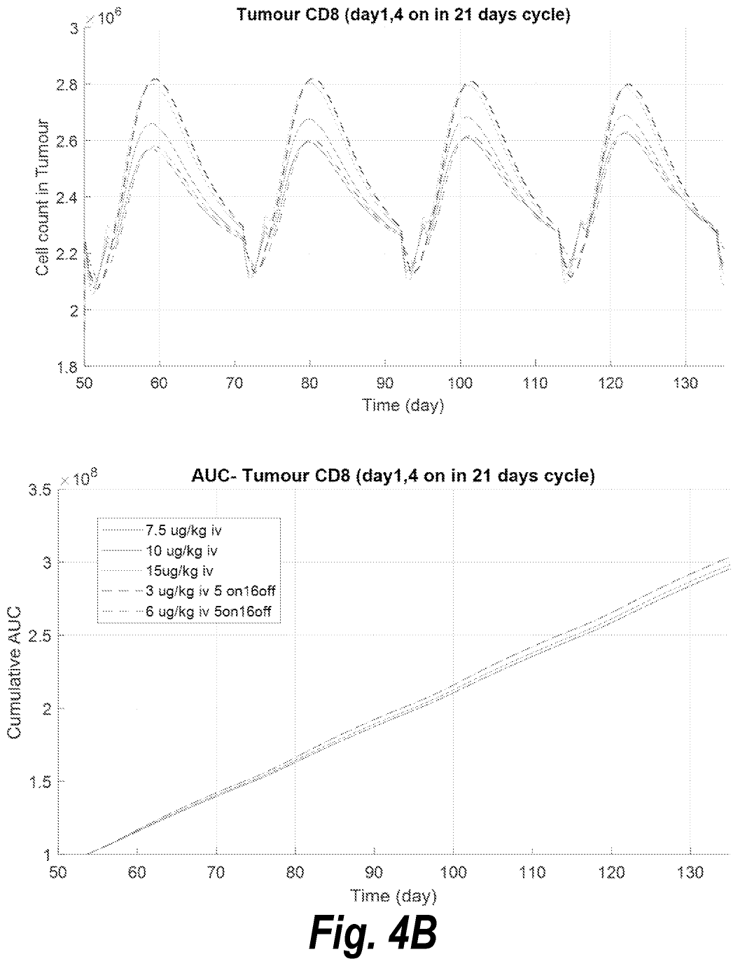

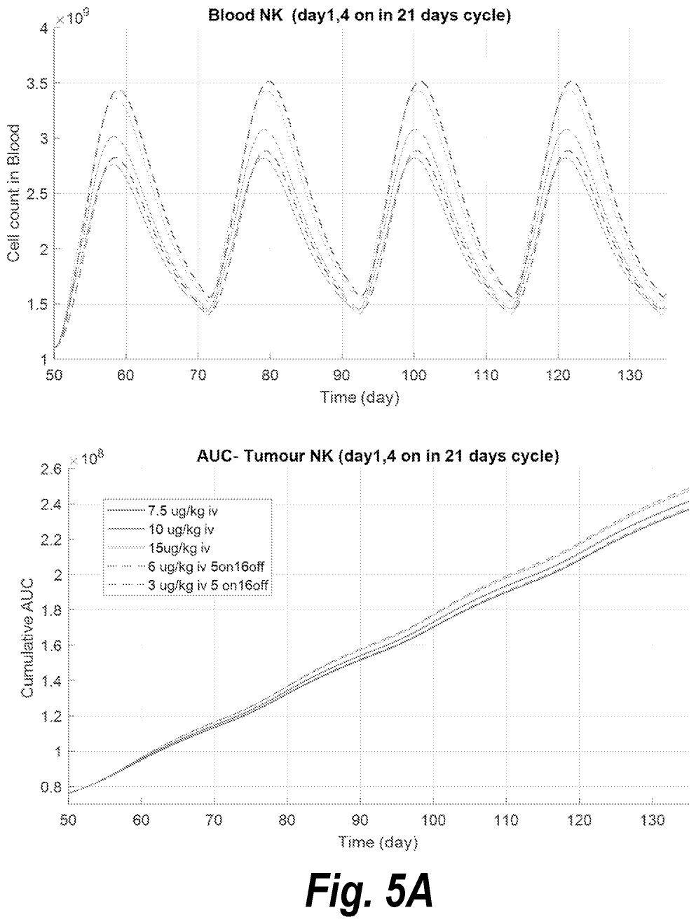

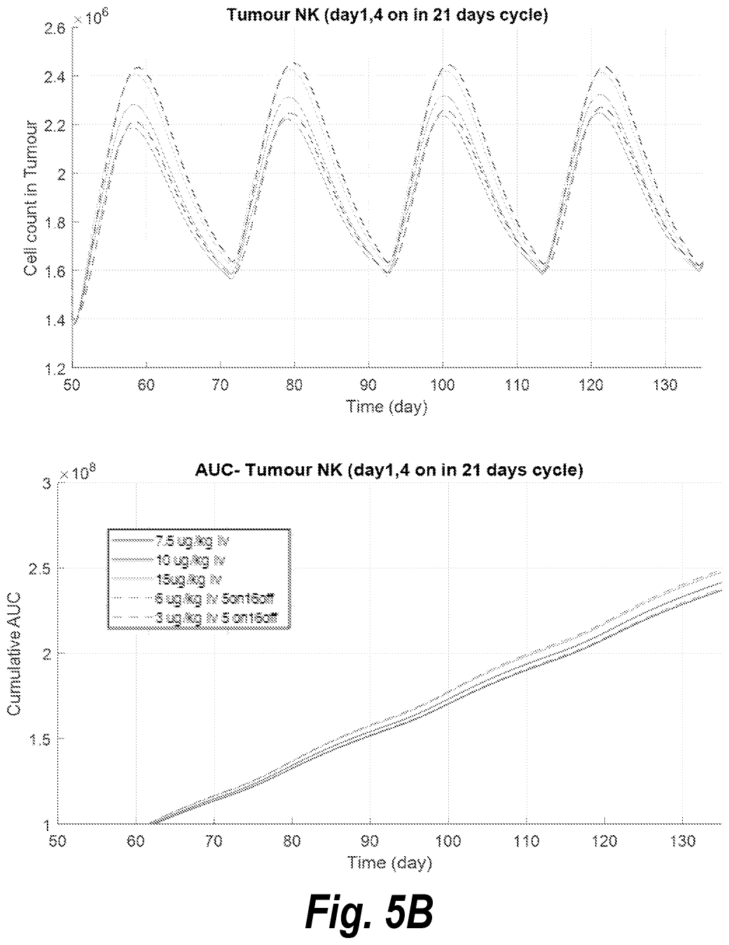

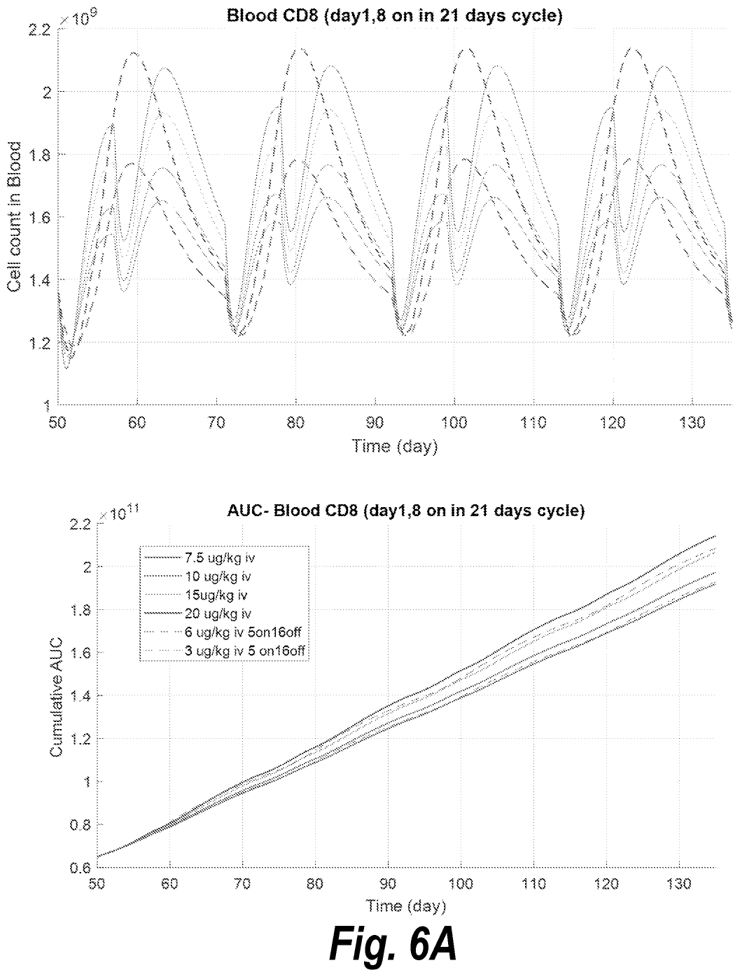

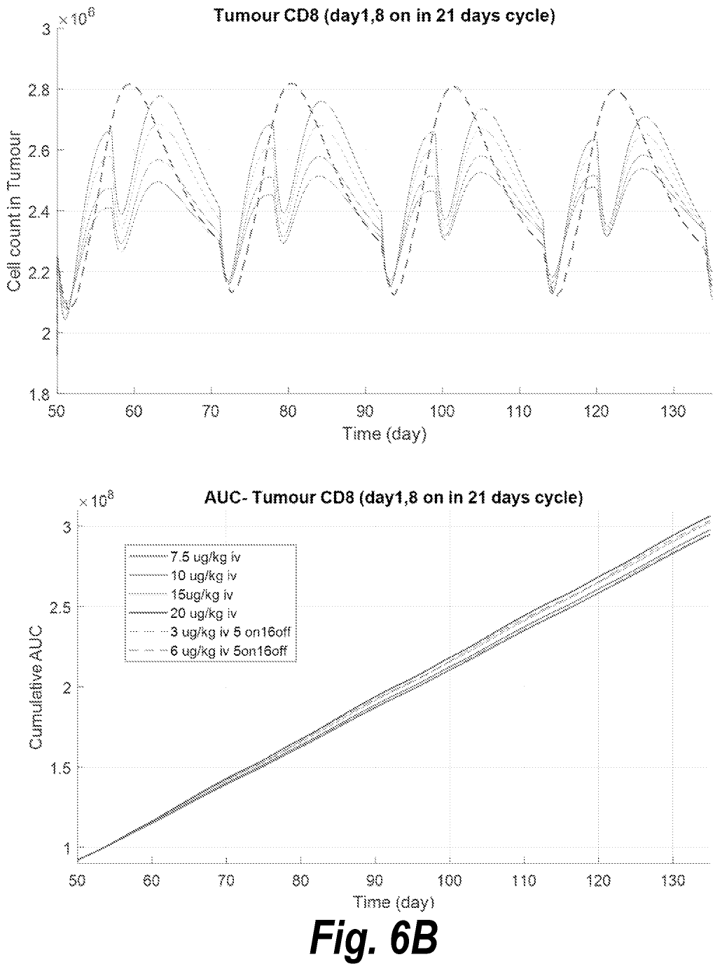

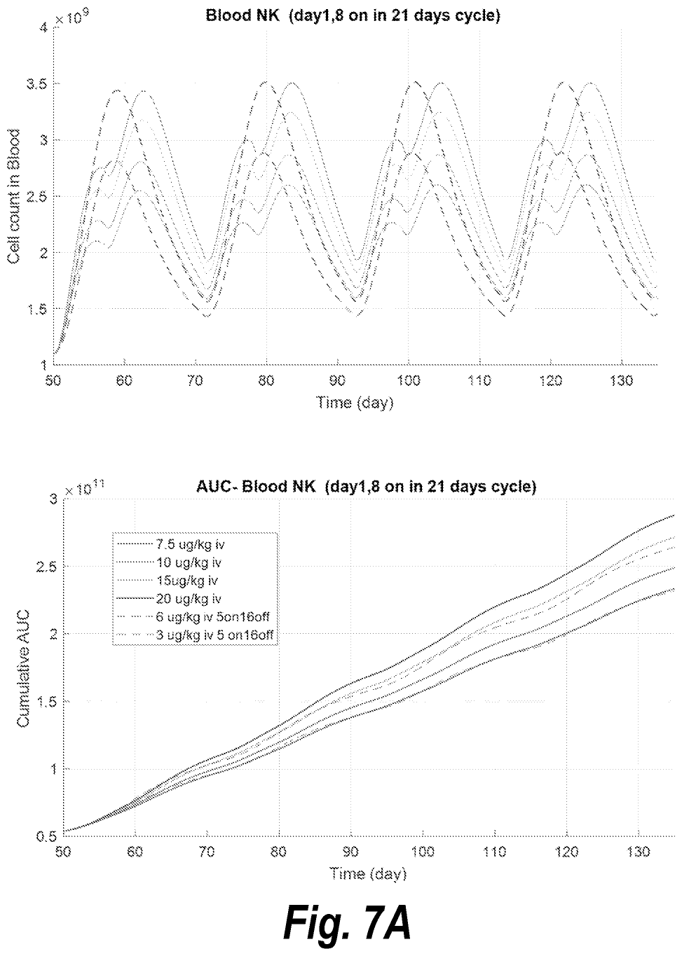

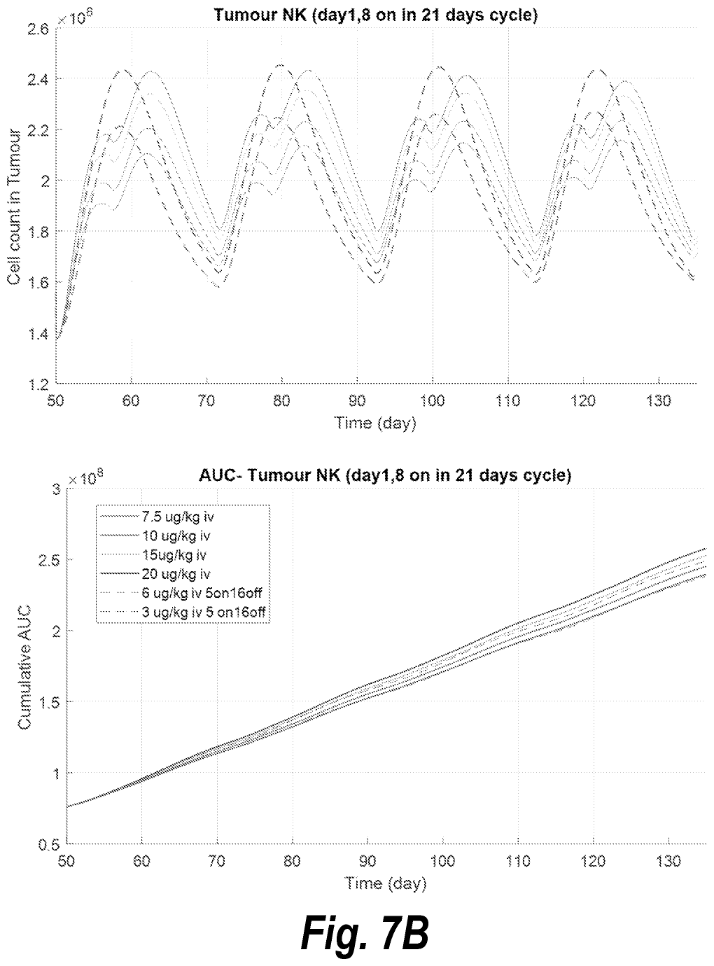

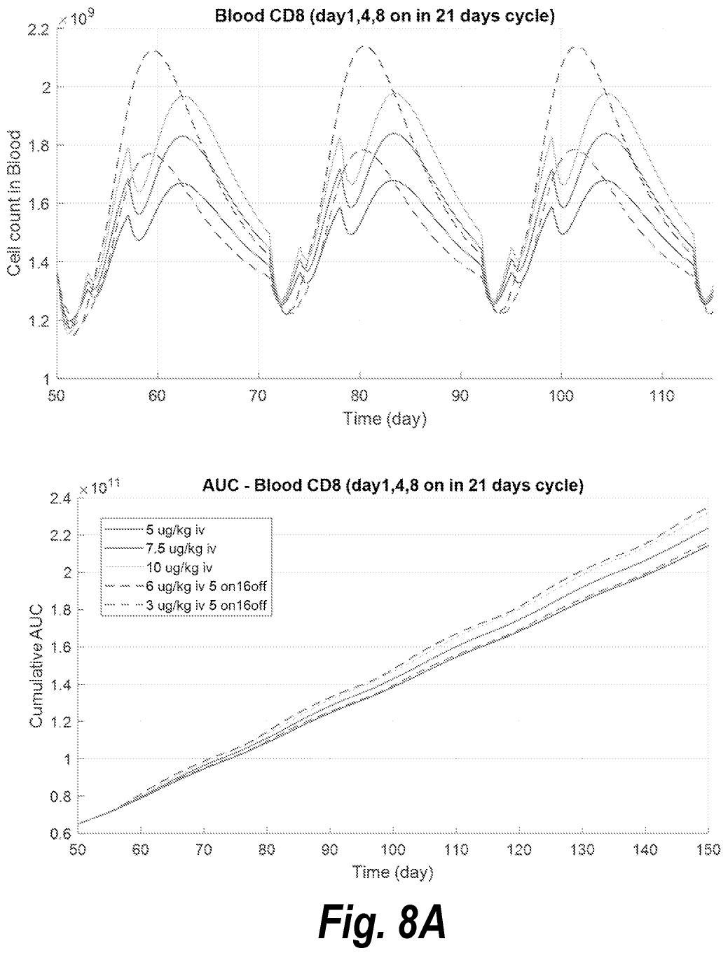

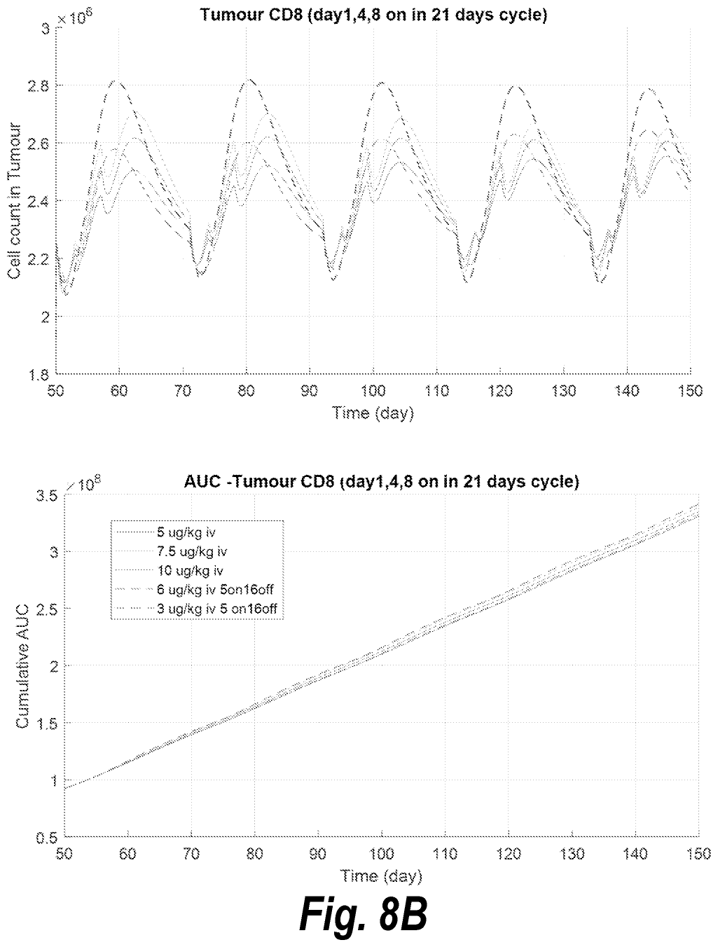

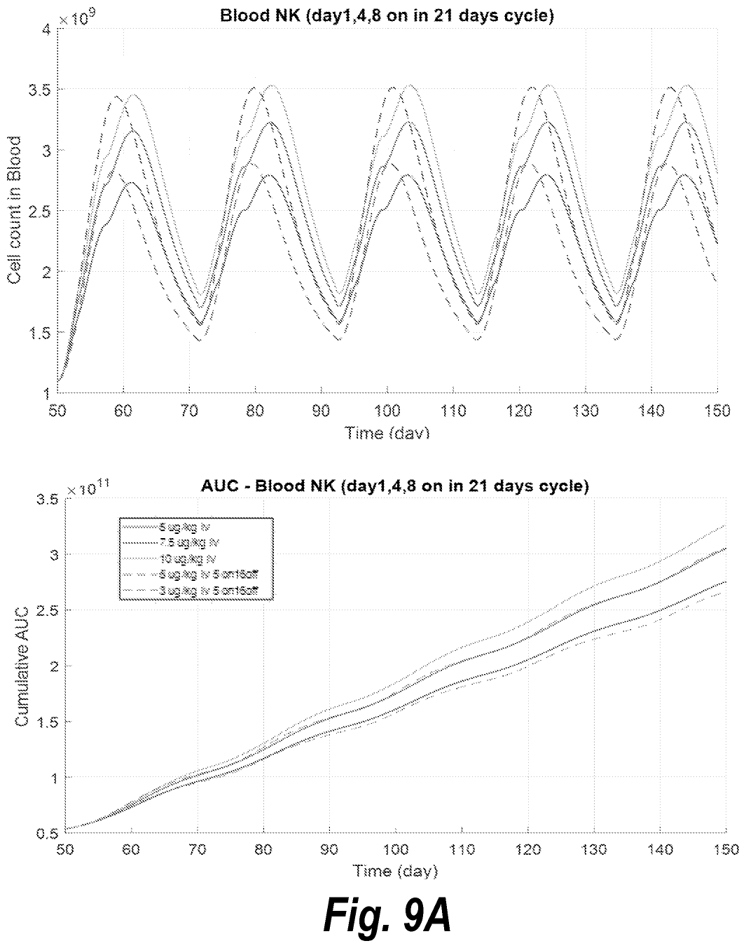

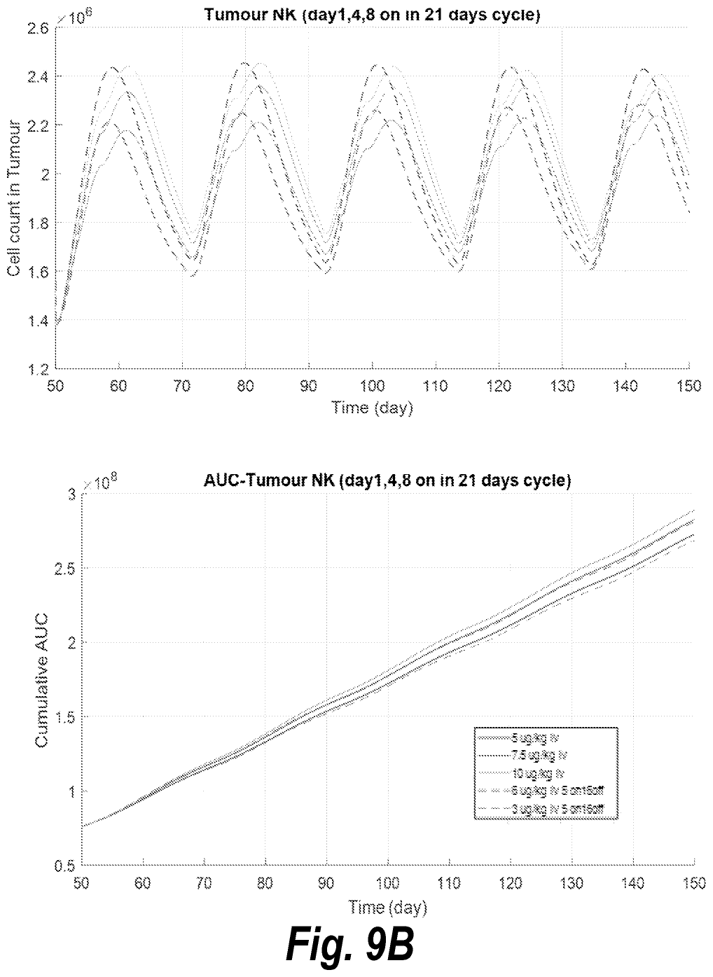

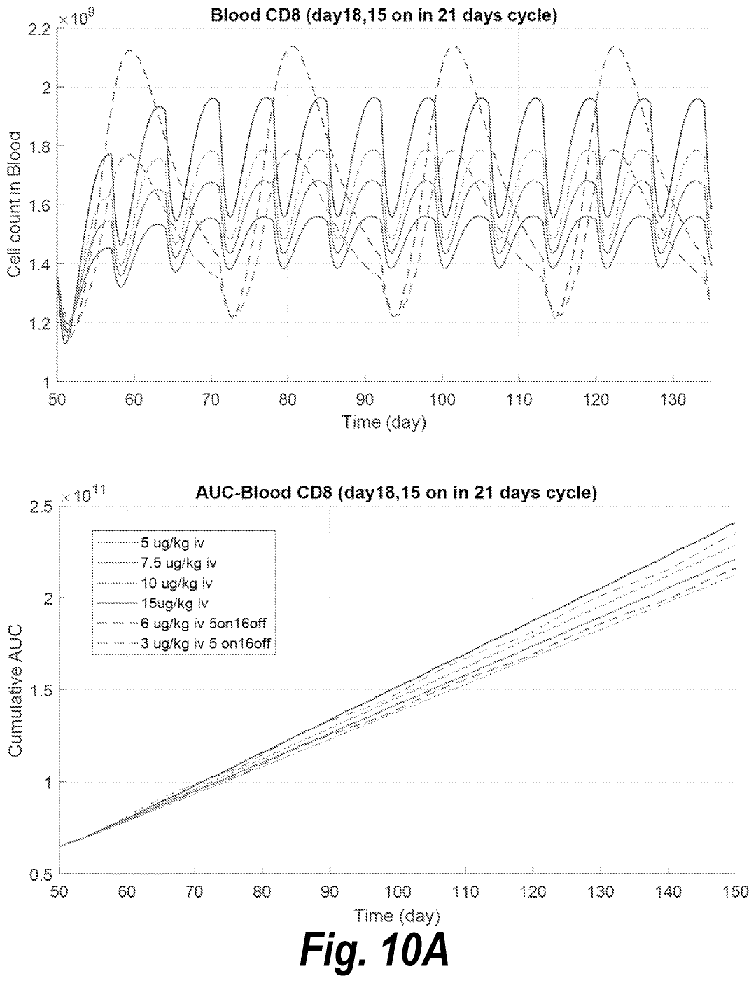

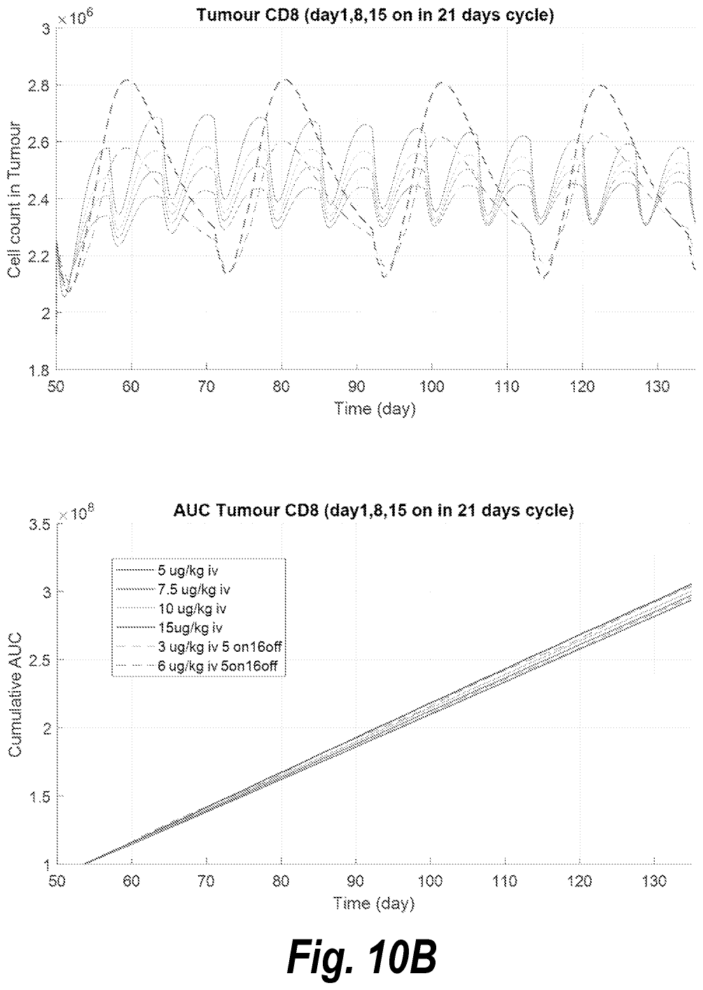

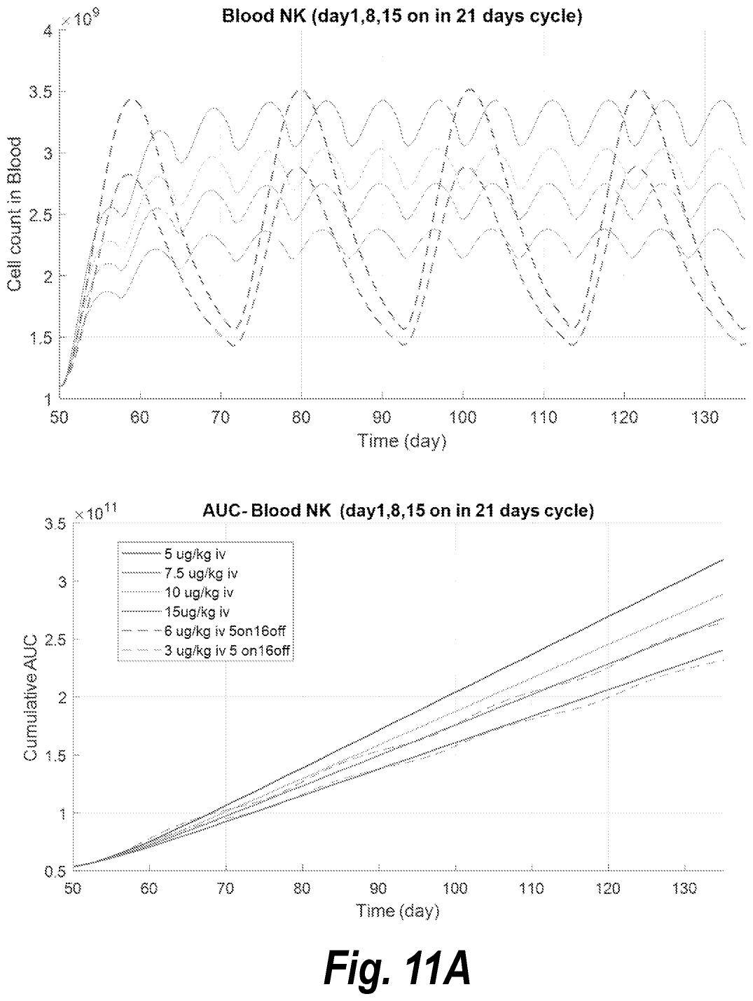

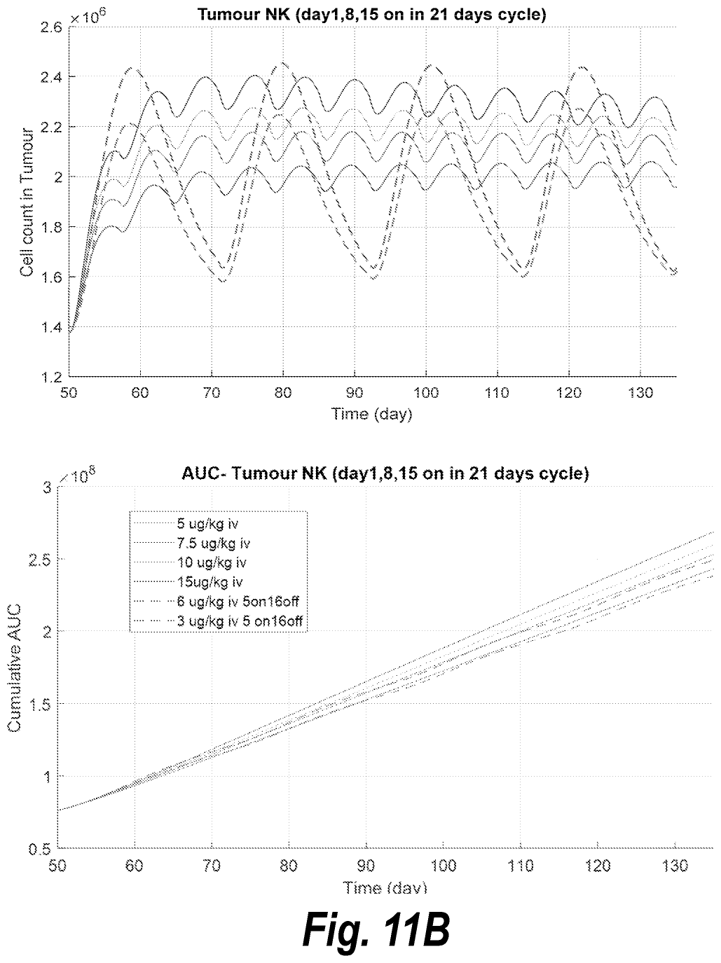

The simulated graphs depicting CD8+ T cell and NK cell numbers in blood and tumor are shown in FIG. 2-11. The graphs start at “Day 50”, which corresponds to the Day 1 dosing recited above for each condition. The results indicate that each of the new less-frequent administration schemes showed similar or better activity than the previous 3 μg/kg or 6 μg/kg qdx5 administration scheme. This was observed for the maximum cell count (Rmax) and AUC values in both CD8+ T cells and NK cells, each in both blood and tumor.

### Example 2—Cohort 2 of the ARTISRY-3 Clinical Trial: A Less-Frequent Dosing Regimen of the Fusion Protein of SEQ ID NO: 1 Administered Intravenously as a Monotherapy and in Combination with Pembrolizumab in Subjects with Advanced Solid Tumors

In view of the modeling data to predict a less frequent dosing regimen, a new cohort (Cohort 2) for the ARTISRY-3 clinical trial (NCT04592653) will be added.

The current dose schedule requires patients to receive the fusion protein of SEQ ID NO: 1 via infusion on 5 consecutive days in a 21-day cycle (Q21D), which limits flexibility of dosing and can be burdensome to patients and staff at treating facilities. Longer dosing interval periods can minimize patient burden and reduce risks associated with more frequent administration (e.g., infusion reactions), as well as exposure to communicable diseases (e.g., SARS-CoV-2) associated with visits to hospitals or infusion centers. It is hypothesized that higher doses of the fusion protein of SEQ ID NO: 1 administered at less frequent dosing will be safe and effective.

TABLE 1
List of Abbreviations and Definition of Terms

| Abbreviation or Term | Full Form of Definition |
|---|---|
| ADL | Activities of daily living |
| AE | Adverse event |
| AESI | Adverse event of special interest |
| ALT | Alanine aminotransferase |
| AST | Aspartate aminotransferase |
| AUC | Area under the curve |
| BOIN | Bayesian optimal interval methodology |
| CA | Cancer antigen |
| CDx | Cluster of differentiation x |
| CLS | Capillary leak syndrome |
| CR | Complete response |
| CRS | Cytokine release syndrome |
| CSA | Clinical Study Agreement |
| CTCAE | Common Terminology Criteria for |
| | Adverse Events |
| ctDNA | Circulating tumor DNA |
| DLT | Dose-limiting toxicity |
| DNA | Deoxyribonucleic acid |
| DOR | Duration of response |
| ECG | Electrocardiogram |
| ECOG PS | Eastern Cooperative Oncology Group |
| | Performance Status |
| eCRF | Electronic case report form |
| EOT | End-of-treatment |
| FDA | Food and Drug Administration |
| GCP | Good Clinical Practice |
| IB | Investigator's Brochure |
| ICF | Informed consent form |
| ICH | International Council for Harmonisation |
| | of Technical Requirements |
| | for Pharmaceuticals for Human Use |
| IEC | Independent ethics committee |
| IF | Immunofluorescence |
| IFN | Interferon |
| IHC | Immunohistochemistry |
| IL | Interleukin |
| IL-2R | Interleukin-2 receptor |
| IO | Immuno-oncology |
| irAE | Immune-related adverse event |
| IRB | Institutional review board |
| iRECIST | Immune Response Evaluation Criteria |
| | in Solid Tumors |
| IRR | Infusion-related reaction |
| IV | Intravenous(ly) |
| MedDRA | Medical Dictionary for Regulatory Activities |
| MSI | Microsatellite instable |
| MSI-H | Microsatellite instable-high |
| MSS | Microsatellite stable |
| MTD | Maximum tolerated dose |
| NCI | National Cancer Institute |
| NK | Natural killer |
| NOAEL | No-observed-adverse-effect level |
| NOS | Not otherwise specified |
| ORR | Overall response rate |
| OS | Overall survival |
| PD | Pharmacodynamic(s) |
| PD-1 | Programmed death receptor-1 |
| PD-L1 | Programmed death-ligand 1 |
| PFS | Progression-free survival |
| PK | Pharmacokinetic(s) |
| PR | Partial response |
| QxD | Every x days |
| QxW | Every x weeks |
| RCC | Renal cell carcinoma |
| RECIST | Response Evaluation Criteria in Solid Tumors |
| RP2D | Recommended phase 2 dose |
| SAE | Serious adverse event |
| SAP | Statistical Analysis Plan |
| SRC | Safety review committee |
| TEAE | Treatment-emergent adverse event |
| TMB | Tumor mutation burden |
| TME | Tumor microenvironment |
| TNBC | Triple-negative breast cancer |
| TNF | Tumor necrosis factor |
| T_{regs} | Regulatory T cells |
| ULN | Upper limit of normal |
| USP | US Pharmacopeia |
| USP-NF | US Pharmacopeia-National Formulary |
| v | Version |
| WHO-ATC | World Health Organization Anatomical |
| | Therapeutic Chemical |
| | (classification system) |
| WOCBP | Women of childbearing potential |

### Cohort 2 (Less Frequent IV Dosing)

Dose selection for Cohort 2 (less frequent IV dosing) is supported by preclinical and clinical evidence.

### 1. Preclinical Data:

Various preclinical studies provide sufficient support for testing a higher dosage of the fusion protein of SEQ ID NO: 1 given 1, 2, or 3 times during a 21-day cycle in a clinical setting.
1. Safety assessment for systemic toxicity and nonclinical exposure margins support the dose selection for Cohort 2 (less frequent IV dosing).
   The maximum observed concentration (Cₘₐₓ) and area under the curve during 24 hours (AUC₀₋₂₄ₕ) exposures on Day 1 at the no-observed-adverse-effect level (NOAEL; 0.1 mg/kg/day) from the repeat-dose (28-day) Good Laboratory Practice IV toxicity study with the fusion protein of SEQ ID NO: 1 in mice (using daily dosing) and the 5-week Good Laboratory Practice IV study in cynomolgus monkeys (using a 5-day on, 9-day off cycling dosage regimen) were at or slightly higher than projected clinical exposures in humans after a single administration of 40 μg/kg (Cₘₐₓ of 920 ng/ml and AUC₀₋₂₄ₕᵣ of 3808 ng hr/mL). Additionally, the calculated AUC_{0-21day} exposures at the NOAEL in mice and monkeys from the 28-day and 5-week IV toxicity studies were compared with the estimated human exposure over a 21-day period (AUC_{0-21day}) using the highest planned repeat-dose regimen in humans (25 μg/kg on Day 1 and Day 8). Exposures in animals at the NOAEL were at least 9.5-fold higher than those estimated in humans (5554 ng·hr/mL).
2. According to International Council for Harmonisation of Technical Requirements for Pharmaceuticals for Human Use (ICH) Guidance, the highest dose or exposure tested in the nonclinical studies does not limit the dose escalation or highest dose investigated in a clinical trial in patients with cancer. However, these observations in monkeys and mice suggest adequate exposure multiples exist between the planned clinical doses under evaluation and doses in animals that were well tolerated systemically.
3. Safety assessment for local toxicity was performed. The NOAEL for the definitive 5-week IV monkey study was 0.1 mg/kg/day (cycling dosing regimen) based on systemic toxicity findings. Because there were no adverse effects at IV injection sites in this study, the high dosage of 0.3 mg/kg/day was used for the local tolerability safety assessment. The 0.3 mg/kg/day dosage translated to approximately 0.75 mg/injection site on the basis of an average monkey body weight of approximately 2.5 kg. In humans, the 25 and 40 μg/kg doses translate to approximately 1.5 and 2.4 mg/injection site, which are 2.0- and 3.2-fold higher than the IV NOAEL in cynomolgus monkeys, respectively. In the dose range-finding IV monkey study, no adverse injection site findings were noted at doses of up to 0.6 mg/kg (the highest dose for which histopathology data are available); this dose in monkeys (approximately 1.5 mg/injection site) is equivalent to the 25 μg/kg human dose on a mg/injection site basis and modestly lower than that at the 40 μg/kg human dose. These nonclinical findings, along with the acceptable local tolerability profile of the fusion protein of SEQ ID NO: 1 after IV administration to humans, support the local safety of planned clinical doses of the fusion protein of SEQ ID NO: 1.
4. In addition, less frequent IV dosing of the fusion protein of SEQ ID NO: 1 with higher dose levels could be administered without causing a decrease in body weight while achieving comparable antitumor efficacy based on results of a B16F10 mouse model of lung metastases (Lopes et al. Journal for Immunotherapy of Cancer. 2020: E000673).

### 2. Clinical Data:

In the ongoing ARTISTRY-1 study, the fusion protein of SEQ ID NO: 1 was shown to be generally well tolerated at dosages up to 8 μg/kg/day for monotherapy and up to 6 μg/kg/day when administered in combination with pembrolizumab. Treatment-related adverse events (AEs) were manageable, and those associated with anticipated on-target effects (e.g., fevers, chills, and reductions in leukocyte subsets) were transient. Systemic exposure to the fusion protein of SEQ ID NO: 1 increased with the increasing dose levels evaluated in the ongoing ARTISTRY-1 study. In patients with diverse solid tumors, serum concentrations of the fusion protein of SEQ ID NO: 1 achieved target exposures exceeding the levels needed for NK and CD8+ T cell activation as determined by preclinical pharmacologic studies. Moreover, the clinical administration of the fusion protein of SEQ ID NO: 1 at dosage levels up to 8 μg/kg/day resulted in selective dose-dependent increases in circulating NK and CD8+ T cells, coupled with minimal, non-dose-dependent changes in T_{regs}. Transient, dose-dependent elevations of serum IL-6 levels occurred within a few hours post-dose and were associated with transient fever and chills. The 6 μg/kg/day monotherapy dosage level (monotherapy RP2D) and the 3 μg/kg/day dosage in combination with pembrolizumab both induced the desired mechanistic effects, with AEs that were anticipated on the basis of mechanism of action and were manageable in both the inpatient and outpatient settings.

In ARTISTRY-1, clinical efficacy was observed in patients treated with the fusion protein of SEQ ID NO: 1 monotherapy at the RP2D of 6 μg/kg (Part B) as well as in patients treated with the fusion protein of SEQ ID NO: 1 at 3 μg/kg and 6 μg/kg in combination with pembrolizumab (Part C). On the basis of the hypothesized mechanism of action for the fusion protein of SEQ ID NO: 1, activation and expansion of tumor killing effector cells such as CD8+ T cells and NK cells are used as the biomarkers for predicting clinical efficacy. Therefore, PD modeling was carried out to predict the dose levels with the less frequent IV dosing regimen (e.g., 1 dose, 2 doses, or 3 doses in a 21-day treatment cycle) to achieve the extent of CD8+ T cell and NK cell expansion comparable to that achieved with the fusion protein of SEQ ID NO: 1 at 3 μg/kg and 6 μg/kg given daily for 5 days in a 21-day treatment cycle.

Doses of 15 μg/kg and 30 to 40 μg/kg given once (on Day 1) in a 21-day cycle are predicted to achieve CD8+ T cell and NK cell expansion levels comparable to those achieved with the fusion protein of SEQ ID NO: 1 at 3 μg/kg and 6 μg/kg, respectively, given once daily for 5 days in a 21-day cycle. For the 2 doses in a 21-day cycle regimen, 2 scenarios were assessed: dosing on Days 1 and 8 and dosing on Days 1 and 4. Doses of 10 μg/kg and 20 to 25 μg/kg given twice (on Days 1 and 8) in a 21-day cycle are predicted to achieve CD8+ T cell and NK cell expansion levels comparable to those achieved with the fusion protein of SEQ ID NO: 1 at 3 μg/kg and 6 μg/kg, respectively, given once daily for 5 days in a 21-day cycle. Doses of 10 μg/kg and 15 μg/kg given twice (on Days 1 and 4) in a 21-day cycle are predicted to achieve CD8+ T cell and NK cell expansion levels comparable to those achieved with the fusion protein of SEQ ID NO: 1 at 3 μg/kg and 6 μg/kg, respectively, given once daily for 5 days in a 21-day cycle.

In Part A of ARTISTRY-1, 43 patients have received the fusion protein of SEQ ID NO: 1 as a monotherapy up to 10 μg/kg. To date, at least 7 patients have received the fusion protein of SEQ ID NO: 1 at 10 μg/kg on Day 1 in the ARTISTRY-1 study. All 7 patients tolerated the dosing on Day 1 and no dose-limiting toxicity (DLT) was reported within 24 hours of Day 1 dosing (before dosing on Day 2). After Day 4 of consecutive IV dosing of the fusion protein of SEQ ID NO: 1 at 10 μg/kg, one DLT was reported in a 70-year-old male patient with history of RCC and left nephrectomy. After receiving 4 doses of the fusion protein of SEQ ID NO: 1 in Cycle 1, he developed grade 4 acute kidney injury requiring hemodialysis after hypotension (responsive to fluids) and nonsteroidal anti-inflammatory drug use. This event led to treatment discontinuation for this patient.

Enrollment in the ARTISTRY-1 trial has closed and the safety review committee (SRC) concluded that the MTD was not reached. Dosage level of 8 μg/kg given on Day 1 to 5 of a 21-day cycle (40 μg/kg cumulative dose/21 day) was well tolerated as well and no DLTs were reported.

PD modeling and clinical data from the ARTISTRY-1 study support starting at 10 μg/kg given on Day 1 of a 21-day cycle.

### Cohort 2 Study Objectives

### Primary Objectives:

1) To investigate the safety and tolerability of less frequent IV dosing schedules of the fusion protein of SEQ ID NO: 1 to identify and determine a RP2D of the fusion protein of SEQ ID NO: 1 as a monotherapy.

2) To determine the MTD of the fusion protein of SEQ ID NO: 1 as a monotherapy and in combination with pembrolizumab.

Each primary objective will be evaluated by measuring the incidence of DLT from the first dose through the end of the DLT observation period.

### Secondary Objectives:

1) To evaluate clinical antitumor activity (ORR, DOR).

2) To evaluate the safety and tolerability of the protein fusion of SEQ ID NO: 1 as a monotherapy, and the protein fusion of SEQ ID NO: 1 in combination with pembrolizumab.

3) To evaluate the clinical PK of the protein fusion of SEQ ID NO: 1.

4) To evaluate immunogenicity of the protein fusion of SEQ ID NO: 1.

5) To evaluate the effects of the protein fusion of SEQ ID NO: 1 as a monotherapy and the protein fusion of SEQ ID NO: 1 in combination with pembrolizumab on circulating leukocyte and peripheral blood soluble protein profiles in patients with a variety of advanced, malignant solid tumors.

The secondary objectives will be evaluated as follows:

1) Clinical antitumor activity: ORR and DOR based on RECIST v1.1 and iRECIST.

2) Occurrence, nature, and severity of AEs; absolute values and change from baseline in vital signs, laboratory tests, and ECGs.

3) Concentrations of the fusion protein of SEQ ID NO: 1 in patients' serum throughout the treatment period and descriptive PK parameters.

4) Presence of anti-fusion protein of SEQ ID NO: 1 antibodies in patients' serum throughout the treatment period.

5) Changes in absolute cell numbers (including total T cells, CD8+ T cells, CD56+ cells, and T_{reg} cells) and ratios (including T/T_{reg}, CD8+/T_{reg}, CD56+/T_{reg}) between pretreatment and on-treatment serial peripheral blood samples obtained from patients being treated with the fusion protein of SEQ ID NO: 1 as a monotherapy and with the combination of the fusion protein of SEQ ID NO: 1 plus pembrolizumab.

### Patient Inclusion Criteria

Each patient must meet all of the following inclusion criteria to be qualified to participate in this study:
1. Patient or the patient's legal guardian(s) is willing and able to provide written informed consent before study initiation.
2. Patient is ≥18 years of age.
3. Patient carries a histologically or cytologically confirmed diagnosis of a malignant solid tumor of a type included within this study (see inclusion criterion 4 below) with at least 1 accessible lesion for biopsy.
4. All patients must have advanced solid malignancies as specified below: histologically or cytologically confirmed epithelial tumor of the fallopian tube, peritoneum, or ovaries, cervical cancer, endometrial cancer, non-small cell lung adenocarcinoma, small cell lung cancer, gastric and gastroesophageal junction adenocarcinoma, esophageal cancer (squamous and adeno cell type), pancreatic cancer, biliary tract tumor (including intra- and extrahepatic cholangiocarcinoma, gall bladder, ampullary type), cutaneous melanoma, mucosal melanoma, head and neck squamous cell carcinoma, or metastatic or advanced breast cancer after treatment failure or intolerance of 1 to 3 established indication-specific therapies. Note: Patient must have received 1 to 3 prior FDA-approved targeted therapies. Failure of adjuvant and neoadjuvant therapy is considered 1 line of treatment.
5. All patients must agree to have the MSI status of his/her malignancy evaluated using his/her baseline biopsy.
6. All patients' baseline biopsies must be taken no more than 3 months before Screening and at least 4 weeks after completion of last antineoplastic therapy.
7. Patients must have at least 1 lesion that qualifies as a target lesion on the basis of the Response Evaluation Criteria in Solid Tumors (RECIST) version (v) 1.1, as determined by the treating physician, and not confounded by prior treatment such as radiation. A lesion will not qualify to be a target lesion if it has been previously irradiated. Patients must be able to provide adequate tissue samples for the planned biopsies, which will not affect measurement of tumor size per RECIST v1.1 criteria.
8. Patients must be ambulatory with an Eastern Cooperative Oncology Group Performance Status (ECOG PS) of 0 or 1 and an estimated life expectancy of at least 3 months.
9. Patients must have adequate hematologic reserve, as evidenced by:
   Absolute neutrophil count of ≥1000/μL
   Absolute lymphocyte count of ≥500/μL within 2 weeks of starting investigational product(s)
   Platelet count of ≥75,000/μL without platelet transfusion support within 2 weeks of starting investigational product(s)
   Hemoglobin of ≥9 g/dL (patients may be transfused to this level, if necessary)
10. Patients must have adequate hepatic and renal function, as evidenced by:
   Aspartate aminotransferase (AST) and alanine aminotransferase (ALT) values ≤3×the upper limit of normal (ULN; ≤5×the ULN if the liver is known to be involved by metastatic disease)
   Serum total bilirubin values of ≤1.5×the ULN (≤2×ULN for patients with known Gilbert's syndrome)
   Serum creatinine≤1.5× the ULN for the reference laboratory or a calculated creatinine clearance of ≥60 mL/min by the Cockcroft-Gault equation
11. For Part A of Cohort 2, treatment with prior immunotherapy is permitted unless the patient has previously experienced grade ≥3 autoimmune toxicity or drug-related toxicity requiring discontinuation. Patients in Part B of Cohort 2 (less frequent IV dosing) who received prior anti-PD-(L) 1 for at least 3 months may enroll if they had a response of stable disease or better.
12. Patients must have recovered from the side effects/toxicities of any prior chemotherapy, immunotherapy, other prior systemic anticancer treatment, radiotherapy, or surgery (ie, toxicity no worse than grade 1, except as otherwise specified [grade 2 alopecia and treatment-associated peripheral neuropathy are acceptable]).
13. For Part A of Cohort 2, patients who have received prior anti-PD-1 directed therapy must wait at least 4 weeks from last dose of such therapy before the Screening biopsy is collected. Patients who have received other standard or investigational agents must wait at least 5 half-lives or 4 weeks (whichever is shorter); or 4 weeks if the half-life of the investigational agent is not known) before the Screening biopsy is collected. For cytotoxic chemotherapy, patients must wait at least 2 weeks with recovery of myeloid function to baseline levels. Shorter wait periods may be permitted after discussion with the Medical Monitor.
14. Women of childbearing potential (WOCBP) must have a negative pregnancy test (serum or urine) within 7 days of the start of treatment and on Day 1 before administration of the first dose of study drug(s).

### Patient Exclusion Criteria

Each patient must not have any of the following conditions to be qualified to participate in this study.
1. Patient is currently pregnant or breastfeeding, or is planning to become pregnant during the study period.
2. Patients with an active infection or with a fever ≥38.5° C. within 3 days of the first scheduled day of dosing for Cycle 1.
3. Patients with primary central nervous system malignancy.
4. Patients with active or symptomatic central nervous system metastases unless the metastases have been treated by surgery and/or radiation therapy and/or gamma knife, the patient has been tapered to a dose of 10 mg of prednisone (or equivalent) or less of corticosteroids for at least 2 weeks before the first dose, and the patient is neurologically stable. Patients with leptomeningeal disease are excluded.
5. Patients with known hypersensitivity to any components of the fusion protein of SEQ ID NO: 1 or pembrolizumab.
6. Patients with an uncontrolled bleeding disorder
7. Patients who have a prolonged partial thromboplastin time >1.5×ULN and/or an international normalized ratio >1.5. Patients on anticoagulant medication must have normalized clotting times or have undergone appropriate interruption and/or reversal of anticoagulant therapy before undergoing biopsies
8. Patients who require pharmacologic doses of systemic corticosteroids (greater than 10 mg of prednisone daily or equivalent); however, topical, ophthalmologic, and inhalational steroids are permitted
9. Patients with mean QT interval corrected by the Fridericia correction formula values of >470 msec (in females) or >450 msec (in males) at Screening; patients who are known to have congenital prolonged QT syndromes; or patients who are on medications known to cause prolonged QT interval on electrocardiogram (ECG). Patients whose ECGs are equivocal or borderline for QT prolongation must undergo an appropriate cardiology evaluation and consultation and be deemed at low risk for arrhythmias and/or other cardiac events before receiving any investigational product(s)
10. Patients who have active autoimmune disease(s) requiring systemic treatment within the past 2 years or a documented history of clinically severe autoimmune disease that has required chronic or frequent systemic steroids. Replacement therapy (eg, thyroxine, insulin, or physiologic corticosteroid replacement therapy for adrenal or pituitary insufficiency) is allowed. Vitiligo is not exclusionary
11. Patient is known to be positive for HIV and/or have a history of hepatitis B or C infections or is known to be positive for hepatitis B antigen/hepatitis B virus DNA or hepatitis C antibody or RNA. Active hepatitis C is defined by a known positive hepatitis C antibody result and known quantitative hepatitis C virus RNA results greater than the lower limits of detection of the assay
12. Subjects who are investigational site staff members directly involved in the conduct of the trial and their immediate family members, site staff members otherwise supervised by the Investigator, or subjects who are Alkermes or Syneos Health employees directly involved in the conduct of the study (immediate family is defined as a spouse, parent, child, or sibling, whether biological or legally adopted)
13. Patients with a known additional malignancy within 2 years of the start of Screening. Exceptions include stable basal cell carcinoma of the skin, squamous cell carcinoma of the skin that has undergone presumably curative therapy, and appropriately treated in situ cervical cancer. Other exceptions may be made with the approval of the Sponsor's Medical Monitor and the Investigator. Patients with a completely treated prior malignancy with no evidence of disease for ≥2 years are eligible
14. Patients with active autoimmune disease that has required systemic treatment in the past 2 years (i.e., with use of disease-modifying agents, corticosteroids, and/or other immunosuppressive drugs). Replacement therapy (e.g., thyroxine) is not considered an excluded form of systemic treatment
15. Patients who have received a live vaccine within 30 days before first dose of study treatment
16. Patients with underlying chronic lung disease, lung primary or metastatic disease, pleural effusions, and/or interstitial lung disease are excluded, unless biopsy can be safely performed on lesions preferably other than lung lesions and room air oxygen saturation is ≥92%
17. Patient has any other concurrent uncontrolled illness, including mental illness or substance abuse, which may interfere with the ability of the patient to cooperate and participate in the study. Other examples of such conditions would include unstable, poorly controlled, or severe hypertension; clinically significant pericardial effusion; New York Heart Association Class III or IV congestive heart failure; known cardiopulmonary disease, defined as unstable angina, myocardial infarction, or cerebrovascular accident within 6 months of first dose; chronic obstructive pulmonary disease or diabetes mellitus that has required 2 or more hospitalizations in the last year; severe peripheral vascular disease; or recent serious trauma
18. Patients with dyspnea at rest or requiring oxygen therapy
19. Patients with prior solid organ and/or nonautologous hematopoietic stem cell or bone marrow transplants
20. Patients who have received radiotherapy within the last 4 weeks before start of study treatment, with the exception of limited field palliative radiotherapy at the discretion of the Medical Monitor
21. Patients who have received systemic immunomodulatory agents within 4 weeks or 5 half-lives, whichever is shorter, before Cycle 1 Day 1, except as per #22 below. Exceptions may be granted on a case-by-case basis by the Medical Monitor
22. Patients who have received prior IL-2-based or IL-15-based soluble protein therapy at any time in the past are excluded

### Overall Study Design and Plan

This study will be conducted in 2 parts: Part A (monotherapy with the fusion protein of SEQ ID NO: 1), and Part B (combination therapy with the fusion protein of SEQ ID NO: 1 and pembrolizumab).

Cohort 2 (less frequent IV dosing) is a multicenter, open-label, sequential groups study. Cohort 2 (less frequent IV dosing) will consist of 2 parts and will assess IV dosing schedules to identify an optimal less frequent IV dosing schedule for both the fusion protein of SEQ ID NO: 1 as a monotherapy and the fusion protein of SEQ ID NO: 1 in combination with pembrolizumab by investigating the safety and tolerability of the fusion protein of SEQ ID NO: 1 for various doses and schedules.

In this cohort, patients with advanced histologically or cytologically confirmed epithelial tumor of the fallopian tube, peritoneum, or ovaries, cervical cancer, endometrial cancer, non-small cell lung adenocarcinoma, small cell lung cancer, gastric and gastroesophageal junction adenocarcinoma, esophageal cancer (squamous and adeno cell type), pancreatic cancer, biliary tract tumor (including intra- and extrahepatic cholangiocarcinoma, gall bladder, ampullary type), any melanoma (except uveal), cervical cancer, head and neck squamous cell carcinoma, or metastatic or advanced breast cancer after treatment failure or intolerance of to up to 3 established lines of indication-specific therapy will be enrolled.

Cohort 2 (less frequent IV dosing) will follow the BOIN design for dose escalation with modifications to accommodate open enrollment. Open enrollment is defined as slot allocation with timeframe for enrollment at all open sites and maximizes utilization of patient resources. An SRC will be established comprising the Principal Investigators, Sponsor's Medical Monitor, Sponsor's Global Safety Officer, and additional representatives of the study team. The SRC will review the safety data from Cohort 2 (less frequent IV dosing) and other relevant information to support the dose escalation/de-escalation decision as well as starting or stopping schedule(s).

Intrapatient dose escalation is permitted after selection of the less frequent IV dosing RP2D is complete in Cohort 2 (less frequent IV dosing) and consultation with Sponsor.

### Part A (Monotherapy)

In Part A, 1 to 3 different dosing schedules may be assessed. The dosing schedule of 1 dose per cycle will be assessed first. Two doses per cycle (Day 1 and Day 8 or Day 1 and Day 4) may be assessed depending on the tolerability assessment in 1 dose per cycle schedule. The MTD will be determined for 1 to 2 dosing schedules.

The 3 dosing schedules in Part A are:
Schedule 1: dosing on Day 1 of a 21-day cycle (1 dose per cycle schedule); planned fusion protein of SEQ ID NO: 1 dose levels include 10, 20, 30, 35, and 40 μg/kg.
Schedule 2: dosing on Day 1 and Day 8 of a 21-day cycle (Day 1, Day 8 dose per cycle schedule); planned fusion protein of SEQ ID NO: 1 dose levels include 15, 20, and 25 μg/kg.
Schedule 3: dosing on Day 1 and Day 4 of a 21-day cycle (Day 1, Day 4 dose per cycle schedule); planned fusion protein of SEQ ID NO: 1 dose levels include 10, 15, and 20 μg/kg.
   The maximum sample size is 30 patients per schedule.

Schedule 1 (1 dose per cycle) of the fusion protein of SEQ ID NO: 1 will be tested first, starting with a dose of 10 μg/kg administered on Day 1 of a 21-day cycle and escalating to doses of 20, 30, 35, and 40 μg/kg. Dose levels of 15 and/or 25 μg/kg may be opened to facilitate de-escalation if the DLT criteria are met for a dose above either of these respective doses and de-escalation is recommended on the basis of dose escalation/de-escalation rules or by the SRC. Up to 6 patients may be enrolled at the 2 lower doses of this schedule (the fusion protein of SEQ ID NO: 1 at 10 and 20 μg/kg). If at least 1 patient has completed the DLT evaluation period without experiencing a DLT, the subsequent patient will be enrolled at the next dose level, first starting from the fusion protein of SEQ ID NO: 1 at 10 to 20 μg/kg and then from the fusion protein of SEQ ID NO: 1 at 20 to 30 μg/kg. Up to 9 patients may be enrolled at each of the higher doses in this schedule (i.e., the fusion protein of SEQ ID NO: 1 at 30, 35, and 40 μg/kg). At least 3 patients must have complete DLT information (completed DLT evaluation and confirmation based on the definitions described in at dose levels ≥30 μg/kg to allow any further dose de-escalation decisions.

More frequent dosing schedule(s) (Schedule 2 and/or Schedule 3) may be opened after the MTD is determined or all expected dose levels are considered tolerable for the Day 1 schedule, especially when the MTD in the Day 1 schedule is below the highest planned dose level and the highest planned dose level cannot be tested, or earlier on the basis of SRC recommendation. In addition, if Schedule 1 assessment reaches the maximum tolerable dose without reaching the MTD, the decision to open Schedule 2 or 3 for an alternative twice-per-cycle dosing will be made in collaboration with the SRC. The second dose escalation will be conducted for Schedules 2 or 3, with dose levels starting at approximately half of the MTD from Schedule 1 and escalated to the highest dose planned for the selected schedule.

For all schedules, additional higher dose levels, if needed, may continue in increments of 5 μg/kg from the highest dose, for at least 1 to 2 dose levels.

### Part B (Combination Therapy)

Part B will start when the highest dose for 1 of the 3 Part A dosing schedules is being tested and a decision is made to stop testing additional schedules. Combination dose will start at a dose below the highest dose level being tested in Part A and may escalate to the highest dose(s) if the highest dose(s) is considered tolerated as monotherapy. A total of 12 to 15 patients will be enrolled in Part B of Cohort 2 (less frequent IV dosing) following BOIN design guidelines. Dose de-escalation to a dose level below the current dose will be considered if the tested combination dose is not tolerated. For Part B, the lowest fusion protein of SEQ ID NO: 1 dose that may be tested in combination with pembrolizumab is 15 μg/kg (if Schedule 1 is selected) or 10 μg/kg (if Schedule 2 or Schedule 3 is selected). All patients in Part B of Cohort 2 will receive pembrolizumab at an approved dosage of 200 mg Q3W.

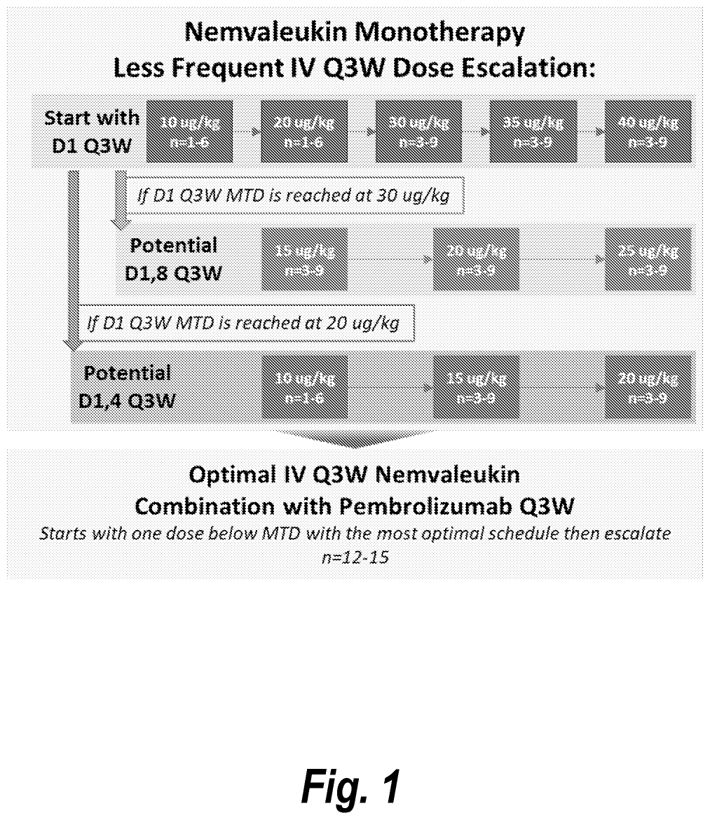

A schematic of the Cohort 2 study design is provided in FIG. 1.

### Dose-Limiting Toxicities

DLTs will be defined by any of the following events that are observed during the interval from Cycle 1, Day 1 through Cycle 1, Day 21 and are deemed as possibly, probably, or definitely related to the fusion protein of SEQ ID NO: 1. The DLT period is defined as duration from first dose to end of first cycle (21 days); however, a longer follow-up may be required to confirm a DLT. To ensure patient safety, study enrollment will be paused (sites will be informed via email or during regularly scheduled team meetings) when 3 or more participants have incomplete DLT information.

### DLT Definitions:

Hematologic: any grade 4 drug-related hematologic AEs that are clinically significant except:
   Grade 4 lymphopenia lasting ≤7 days in duration and not associated with clinical manifestations;
Nonhematologic: any grade ≥3 nonhematologic AEs, including blood chemistry/electrolyte imbalances or abnormalities, deemed related to study drug that do not resolve to grade 2 or lower within 7 days, including:
   Drug-induced liver injury of any grade;
   Any grade ≥2 myocarditis;
   Any grade ≥3 hypotension/cytokine release syndrome occurring within 24 to 48 hours after study drug administration, lasting >72 hours, and manageable with supportive treatment;
   Any grade ≥3 capillary leak syndrome;
   Any treatment-related event leading to discontinuation or requiring longer than 7 days' delay in starting Cycle 2;
      Exception: fatigue, nausea, vomiting, and diarrhea that can be medically managed and alopecia. Tumor flare reported as a related AE will not be considered a DLT.
         Patients may remain on treatment until criteria for treatment discontinuation are met.
         Dose Escalation and/or De-Escalation

The dose escalation and/or de-escalation decision, or declaring a dose unsafe, will be made in collaboration with the study SRC on the basis of the following BOIN guidelines.

Data from the current dose are used to determine the dosing schedule assignment for a new patient entering the study. This decision is based on the estimated probability of DLT on the current dose. Let \hat{π}_{c} be the observed proportion of DLTs on the current dose. The recommended dose is selected as follows:
If \hat{π}_{c}˜≥0.358, the study de-escalates. The recommended dose to treat the next patient is one dose level below the current dose.
If \hat{π}^{c}≤0.236, the study escalates. The recommended dose to treat the next patient is one dose level above the current dose.
Otherwise, the recommended dose to treat the next patient is the current dose.

The lower and upper boundary is derived using a noninformative prior following the calculation in BOIN design to minimize of the probability of incorrect dose assignment.

Dose level is considered unsafe if Pr (π_{d}>0.3|Data)≥0.95 (ie, there is a 95% probability that the dose level lies above the MTD), where na is the probability of DLT on dose d. This calculation is done using independent beta-binomial models for each dose level with a Beta(0.5, 0.5) for prior distribution.

If the escalation/de-escalation rules recommend de-escalating from the lowest dose level and fewer than 3 patients are assigned to that dose, patients may continue to be assigned to the lowest dose level. The purpose of this rule is to ensure that sufficient data are collected on the lowest dose level. The DLT decision criteria are summarized in Table 2 below.

TABLE 2
DLT Decision Criteria

| | Number of DLT-evaluable patients | | | | | | | | |
|---|---|---|---|---|---|---|---|---|---|
| | in the current cohort total | | | | | | | | |
| | 1 | 2 | 3 | 4 | 5 | 6 | 7 | 8 | 9 |
| Maximum DLTs to | — | — | 0 | 0 | 1 | 1 | 1 | 1 | 2 |
| escalate | | | | | | | | | |
| Minimum DLTs to | 1 | 1 | 2 | 2 | 2 | 3 | 3 | 3 | 4 |
| de-escalate | | | | | | | | | |
| Minimum DLTs to | — | 2 | 3 | 3 | 4 | 4 | 5 | 5 | 6 |
| declare a dose | | | | | | | | | |
| unsafe | | | | | | | | | |

Abbreviation: DLT = dose-limiting toxicity.

If a dose level is unsafe, all dose levels above that dose are also considered unsafe. New patients cannot be enrolled on an unsafe dose; however, the new patient may be enrolled in the current dose level or at a lower dose level, at the recommendation of the SRC, if maximum patient enrollment in these dose levels has not yet been achieved.

As additional information becomes available, the decision to declare a dose unsafe may change. For example, if there are 3 patients enrolled at a certain dose and 2 patients with complete data have DLTs (2/2), that dose is considered unsafe. However, when the third patient completes the DLT observation period, if that patient does not experience a DLT (2/3), the dose will no longer be unsafe.

The MTD is determined when enrollment in the study is stopped and every patient has complete DLT information. The MTD is determined using a Bayesian logistic regression.

### Fusion Protein of SEQ ID NO: 1 Dosing and Administration

A patient is considered enrolled after he or she has provided written informed consent and completed all Screening assessments and provided a pretreatment biopsy. Study drug(s) should begin as close as possible to the date/time of enrollment but no later than 72 hours afterward.

In Cohort 2 (less frequent IV dosing), the fusion protein of SEQ ID NO: 1 will be administered via a 30-minute (−5 minutes/+10 minutes) IV infusion on assigned schedule day(s). Each cycle will be 21 days in length. In Part B, the fusion protein of SEQ ID NO: 1 will be administered as a 30-minute IV infusion of the assigned dose, followed by a 30-minute IV infusion (−5 minutes/+10 minutes) of pembrolizumab 200 mg. If a 30-minute infusion of a dose higher than 10 μg/kg of the fusion protein of SEQ ID NO: 1 is not well tolerated, longer infusion time may be considered if recommended by the SRC (i.e., 60 minutes or 90 minutes). Sites will be notified of this change.

Antipyretics should be administered before the fusion protein of SEQ ID NO: 1. The fusion protein of SEQ ID NO: 1 may be infused via peripheral or central venous access. Patients undergoing treatment with the fusion protein of SEQ ID NO: 1 should have a large-bore peripheral IV catheter placed to facilitate fluid management. Patients who do not have adequate peripheral venous access should have a central venous access device placed.

In Cohort 2 (less frequent IV dosing), patients should be monitored for 6 to 8 hours post infusion after dosing in Cycle 1 and Cycle 2. For patients experiencing AEs that are not manageable/resolved at the end of observation period, then the patients must be hospitalized for overnight observation and supportive care must be administered. From Cycle 3 onwards, the Investigator may reduce the post infusion observation period to 2 hours at their discretion, except for patients who required inpatient observation in first the 2 cycles. For such patients who required inpatient observation in the first 2 cycles, mandatory observation period in future cycles can be reduced to 2 hours after consultation with the Sponsor's Medical Monitor.

Patients receiving investigational treatment with the fusion protein of SEQ ID NO: 1 and pembrolizumab will continue receiving treatment for up to 2 years of total exposure (starting from Cycle 1 Day 1 of this study), if they appear to be deriving clinical benefit (i.e., stable disease or better) and tolerating therapy (whichever is shorter), at the discretion of the treating Investigator. Treatment with the fusion protein of SEQ ID NO: 1 in combination with pembrolizumab will be allowed for up to a maximum of 35 treatments (35 cycles; approximately 2 years) if it is well tolerated and continues to show benefit. Patients receiving the fusion protein of SEQ ID NO: 1 and pembrolizumab combination therapy may continue the fusion protein of SEQ ID NO: 1 as monotherapy without pembrolizumab beyond 35 cycles upon discussion with the Sponsor and if they do not meet any other criteria for discontinuation.

### Pembrolizumab Dosing and Administration

Pembrolizumab will be administered via a 30-minute (−5 minutes/+10 minutes) IV infusion at a dosage of 200 mg q21d for up to 35 cycles or 2 years, as long as patients are deriving clinical benefit (i.e., stable disease or better) and tolerating therapy, in accordance with the prescribing information for pembrolizumab. Patients will be monitored for at least 1 hour for potential acute reactions to pembrolizumab before administration of the fusion protein of SEQ ID NO: 1, as described above.

Pembrolizumab is available as single-dose vials of either lyophilized powder or solution and should be managed according to the prescribing information for pembrolizumab.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A method of treating cancer in a subject comprising administering to the subject a fusion protein comprising a sequence at least 80% identical to SEQ ID NO: 1, wherein the subject is administered at least a first dose of the fusion protein and a second dose of the fusion protein on non-consecutive days.

2. The method of claim 1, wherein the fusion protein is administered at doses of about 1 μg/kg to about 60 μg/kg.

3.-4. (canceled)

5. The method of claim 1, wherein the first dose and the second dose are the same.

6.-7. (canceled)

8. The method of claim 1, wherein the subject is administered the first dose of the fusion protein on day 1 and the second dose of the fusion protein on any one of day 3, day 4, day 5, day 6, day 7, day 8, day 9, day 10, day 11, day 12, day 13, day 14, day 15, day 16, day 17, day 18, day 19, day 20, or day 21.

9. The method of claim 1, wherein the subject is administered the first dose of the fusion protein on day 1 and the second dose of the fusion protein on day 4.

10. The method of claim 1, wherein the subject is administered the first dose of the fusion protein on day 1 and the second dose of the fusion protein on day 8.

11.-19. (canceled)

20. The method of claim 1, wherein the subject is administered a first dose of the fusion protein of about 10 μg/kg to about 30 μg/kg on day 1, and a second dose of the fusion protein of about 10 μg/kg to about 30 μg/kg on any one of day 3, day 4, day 5, day 6, day 7, day 8, day 9, day 10, day 11, day 12, day 13, day 14, day 15, day 16, day 17, day 18, day 19, day 20, or day 21.

21.-34. (canceled)

35. A method of treating cancer in a subject comprising administering to the subject patient a fusion protein comprising a sequence at least 80% identical to SEQ ID NO: 1, wherein the subject is administered at least a first dose of the fusion protein, wherein the subject is not administered a dose of the fusion protein at least one day prior to the first dose and the subject is not administered a dose of the fusion protein one day after the first dose.

36. A method of treating cancer in a subject comprising administering to the patient a fusion protein comprising a sequence at least 80% identical to SEQ ID NO: 1, wherein the subject is administered a single dose of the fusion protein once every week (Q1W), once every 2 weeks (Q2W), once every 3 weeks (Q3W), or once every 4 weeks (Q4W).

37. The method of claim 36, wherein the fusion protein is administered at a dose of about 10 μg/kg to about 60 μg/kg.

38. The method of claim 36, wherein the fusion protein is administered at a dose of about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, or about 60 μg/kg.

39.-46. (canceled)

47. The method of claim 1, wherein each dose is administered by intravenous injection or infusion.

48. The method of claim 1, wherein each dose is administered by intravenous injection or infusion over a period of about 30 minutes, about 60 minutes, or about 90 minutes.

49. The method of claim 1, further comprising administering to the subject a therapeutically effective amount of a therapeutic agent.

50. The method of claim 49, where the therapeutic agent is a PARP inhibitor, an immune checkpoint inhibitor, a cytotoxic agent, or a chemotherapeutic agent.

51. The method of claim 49, wherein the therapeutic agent is an immune checkpoint inhibitor.

52. (canceled)

53. The method of claim 51, wherein the immune checkpoint inhibitor is pembrolizumab.

54.-55. (canceled)

56. The method of claim 53, wherein the pembrolizumab is administered in an amount of 200 mg by intravenous injection or infusion.

57.-59. (canceled)

60. The method of claim 1, wherein the cancer being treated is a solid tumor.

61. The method of claim 60, wherein the solid tumor is a carcinoma, sarcoma or lymphoma.

62. The method of claim 1, wherein the cancer is renal cell carcinoma (RCC), melanoma, mucosal melanoma, breast cancer, pancreatic cancer, prostate cancer, non-small cell lung cancer, liver cancer, colon and rectal cancer, bladder cancer, cervical cancer, ovarian cancer, thyroid cancer, esophageal cancer, oral cancer, mesothelioma, non-melanoma skin cancer, or gastric cancer.

63. The method of claim 1, wherein the cancer is a blood cancer.

64. The method of claim 63, wherein the blood cancer is leukemia, non-Hodgkin lymphoma, Hodgkin lymphoma, or multiple myeloma.

65. A pharmaceutical composition comprising a fusion protein comprising a sequence at least 80% identical to SEQ ID NO: 1, wherein the pharmaceutical composition comprises a unit dosage of about 1 μg/kg to about 60 μg/kg.

66.-67. (canceled)


SEQUENCE LISTING (1 sequence)

SEQ ID NO: 1 - protein, 303 aa, synthetic construct
SKNFHLRPRDLISNINVIVLELKGSETTFMCEYADETATIVEFLNRWITFSQSIISTLTGGSSSTKKTQLQLEHLLLDLQMILNGINNYKNPKLTRMLTFKFYMPKKATELKHLQCLEEELKPLEEVLNLAQGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG